(19) United States
(12) Patent Application Publication
(10) Pub. No.: US 2025/0084112 A1
(43) Pub. Date: Mar. 13, 2025

(54) Chemical Compounds Useful for Inhibiting Nav1.8 Voltage-Gated Sodium Channels and Treating Nav1.8 Mediated Diseases

(71) Applicant: GlaxoSmithKline Intellectual Property Development Limited, Stevenage (GB)

(72) Inventors: Jie GUANG, Collegeville, PA (US); David Glenn WASHBURN, Collegeville, PA (US)

(73) Assignee: GlaxoSmithKline Intellectual Property Development Limited, Stevenage (GB)

(21) Appl. No.: 18/256,989

(22) PCT Filed: Dec. 16, 2021
(86) PCT No.: PCT/EP2021/089101

§ 371 Date: Jun. 12, 2023

(51) Int. Cl. C07F 9/6561; A61K 31/675

(52) U.S. Cl. CPC C07F 9/6561; A61K 31/675

(57) ABSTRACT

Compounds of formula (1) are described, wherein each of the variable groups is as defined in the specification. Also described are pharmaceutical compositions containing a compound of formula (1), and uses of the compounds and pharmaceutical compositions for inhibiting Naᵥ1.8 voltage-gated sodium channels and treating Nav1.8 mediated diseases, disorders, and conditions, such as pain and pain-associated diseases, disorders, and conditions and cardiovascular diseases, disorders, and conditions.

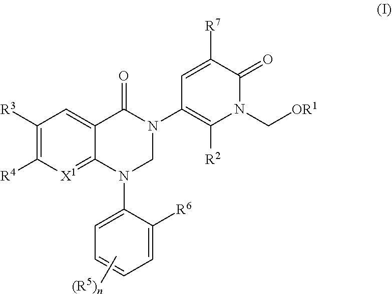

### FIELD OF THE INVENTION

The invention relates to Naᵥ1.8 inhibitor compounds or pharmaceutically acceptable salts or tautomer forms thereof, corresponding pharmaceutical compositions or formulations, methods or processes of compound preparation, methods, compounds for use in, uses for and/or combination therapies for treating pain and pain-associated diseases, disorders and conditions, and cardiovascular diseases, disorders, and conditions.

### BACKGROUND OF THE INVENTION

Pain is a protective mechanism by which animals avoid potential tissue damage, however there are numerous disease indications in which pain outlives its usefulness and becomes a disabling burden. Indications in which pain outlives its usefulness can be broadly categorized as those in which nerve damage or injury is the trigger (neuropathic pain), those in which an inflammatory response or metabolic dysregulation sensitizes the pain response (inflammatory pain) and those in which an injury or surgical procedure results in a short term elevation of pain response (post-operative/ambulatory pain).

Voltage-gated sodium channels underlie electrical signaling in all excitable tissues by setting the threshold and underlying the upstroke of action potentials. There are nine distinct isoforms of voltage-gated sodium channels. Those designated Naᵥ1.1, Naᵥ1.7, Naᵥ1.8 and Naᵥ1.9 are principally expressed on peripheral nerves where they control neuronal excitability. Naᵥ1.5 is the principle sodium channel isoform expressed in cardiac myocytes, Naᵥ1.4 is expressed and functions in skeletal muscle, whilst Naᵥ1.1, Naᵥ1.2, Naᵥ1.3 and Naᵥ1.6 are widely expressed in the central nervous system (CNS) and to an extent in the peripheral nervous system. The principal role of these nine voltage-gated sodium channels is comparable in that they control sodium influx into cells but their biophysical properties varies which greatly influences the physiological profile of their respective cell type (Catterall, 2012).

Currently, non-selective sodium channel inhibitors are utilized clinically as anti-arrhythmic and anti-seizure therapies, these include lidocaine, carbamazepine, amitriptyline and mexiletine. However, as these agents exhibit a lack of selectivity between the different sodium channel isoforms, their therapeutic utility is greatly reduced due to adverse side effects, largely mediated by activity in the CNS and heart. This has stimulated efforts to develop novel medicines which are selective for specific sodium channel isoforms in order to avoid side effects in the CNS and cardiovascular system.

The Naᵥ1.8 channel is expressed in neurons of the dorsal root ganglia (DRG) and highly expressed in the small diameter neurons of this tissue which form pain sensing C- and Aδ-nerve fibers (Abrahamsen, 2008; Amaya, 2000; Novakovic, 1998). The channel was proposed as a therapeutic target for analgesia as soon as it was originally cloned from rat DRG (Akopian, 1996) due to its prominent physiological role in this tissue type and restricted expression profile. Naᵥ1.8 was subsequently identified, cloned and characterized from human DRG tissue (Rabart 1998). The closest molecular relative of Naᵥ1.8 is Naᵥ1.5 which shares a sequence homology of ˜60%. Naᵥ1.8 was previously known as SNS (sensory neuron sodium channel), PN3 (peripheral nerve sodium channel type 3), and as it exhibits characteristic pharmacological properties in its resistant to block by tetrodotoxin, it is also described as a TTX-resistant sodium channel.

Support for Naᵥ1.8 as a therapeutic target for pain indications comes from several sources. Naᵥ1.8 has been shown to conduct the majority of current during upstroke of the action potential in DRG neurons (Blair & Bean, 2002) and due to its rate of re-priming is also critical for the ability of these neurons to fire repetitively (Blair and Bean, 2003). Increased expression and function of Naᵥ1.8 has been reported in response to painful stimuli such as inflammatory mediators (England 1996 & Gold 1996), nerve damage (Roza 2003 & Ruangsri 2011), and within painful neuromas (Black 2008 & Coward 2000). Knockout of the gene encoding Nav1.8 in mice resulted in a reduced pain phenotype in particular to inflammatory challenges (Akopian 1999). Knockdown of the mRNA encoding Naᵥ1.8 also resulted in reduced painful phenotypes in rodent models, particularly in neuropathic models (Lai 2002). Pharmacological intervention via selective small molecule inhibitors has demonstrated efficacy in rodent models of inflammatory pain as well as neuropathic pain (Jarvis 2007 & Payne 2015). Supporting genetic evidence for Naᵥ1.8 is also present in patients with chronic neuropathic pain where multiple gain of function mutations has been reported to be causative in episodic painful neuropathies and small fiber neuropathies (Faber 2012, Han 2014 & Eijkenboom 2018).

### SUMMARY OF THE INVENTION

Accordingly, there is a need for the development of novel compounds, particularly Naᵥ1.8 inhibitor compounds that have improved solubility and are thus more advantageous for alternative routes of administration, such as intravenous administration. The invention satisfies this need by providing prodrugs of compounds with Naᵥ1.8 inhibitory activity and uses of such prodrugs in the treatment of pain and pain associated diseases, disorders, and conditions, and in the treatment of cardiovascular, diseases, disorders, and conditions. The prodrugs of the invention have improved solubility as compared to their respective parent compounds, and thus can be useful for intravenous (IV) administration and treatment of pain and pain associated diseases, disorders, and conditions in which IV administration may be beneficial or preferred, such as in the treatment of acute pain.

In one aspect, the invention relates to a compound of formula (I):

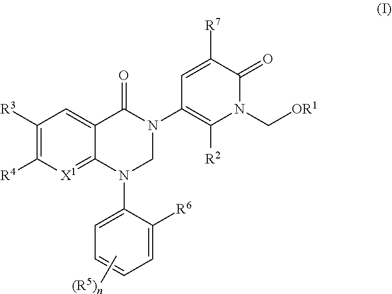

or a pharmaceutically acceptable salt thereof,
wherein:
   X¹ is N or CH;
   R¹ is —PO(OH)₂;
   R² is hydrogen, -(C₁₋₆)alkyl, —NR^{a}R^{b}, halo, or -(C₁₋₆)haloalkyl;
   each of R³ and R⁴ is independently hydrogen, halo, cyano, —NR^{a}R^{b}, -(C₁₋₆)alkyl, -(C₁₋₆)haloalkyl, —O-(C₁₋₆)alkyl, or —O-(C₁₋₆)haloalkyl;
   each R⁵ is independently halo, -(C₁₋₆)alkyl, —O(C₁₋₆)alkyl, or —O(C₁₋₆)haloalkyl;
   R⁶ is hydrogen or -(C₁₋₆)alkyl;
   R⁷ is hydrogen, -(C₁₋₆)alkyl, halo, or -(C₁₋₆)haloalkyl;
   each of R^{a} and R^{b} is independently hydrogen or -(C₁₋₆)alkyl; and
   n is 0, 1, or 2.

In one aspect, the invention relates to a pharmaceutical composition comprising a compound or a tautomer thereof, or a pharmaceutically acceptable salt thereof as defined herein, and a pharmaceutically acceptable excipient.

In one aspect, the invention relates to a method of inhibiting a Naᵥ1.8 voltage-gated sodium channel in a subject in need thereof, the method comprising administering to the subject a compound or a tautomer thereof, or a pharmaceutically acceptable salt thereof as defined herein or a pharmaceutical composition as defined herein.

In one aspect, the invention relates to a method of treatment of pain or a pain-associated disease, disorder, or condition in a subject in need thereof, the method comprising administering to the subject a therapeutically effective amount of a compound, or a tautomer thereof, or a pharmaceutically acceptable salt thereof as defined herein or a pharmaceutical composition as defined herein.

In one aspect, the invention relates to a method of treatment of atrial fibrillation in a subject in need thereof, the method comprising administering to the subject a therapeutically effective amount of a compound, or a tautomer thereof, or a pharmaceutically acceptable salt thereof as defined herein or a pharmaceutical composition as defined herein.

In one aspect, the invention relates to a compound, or a tautomer thereof, or a pharmaceutically acceptable salt thereof as defined herein or a pharmaceutical composition as defined herein for use in treatment of pain or a pain-associated disease, disorder, or condition.

In one aspect, the invention relates to a compound, or a tautomer thereof, or a pharmaceutically acceptable salt thereof as defined herein or a pharmaceutical composition as defined herein for use in treatment of atrial fibrillation.

In one aspect, the invention relates to use of a compound, or a tautomer thereof, or a pharmaceutically acceptable salt thereof as defined herein or a pharmaceutical composition as defined herein in the manufacture of a medicament for treatment of pain or a pain-associated disease, disorder, or condition.

In one aspect, the invention relates to use of a compound, or a tautomer thereof, or a pharmaceutically acceptable salt thereof as defined herein or a pharmaceutical composition as defined herein in the manufacture of a medicament for treatment of atrial fibrillation.

In one aspect, the invention relates to a compound, or a tautomer thereof, or pharmaceutically acceptable salt thereof as defined herein, or a pharmaceutical composition as defined herein for use in therapy.

### DETAILED DESCRIPTION OF THE INVENTION

Various publications, articles and patents are cited or described in the background and throughout the specification. Discussion of documents, acts, materials, devices, articles or the like which has been included in the present specification is for the purpose of providing context for the disclosure. Such discussion is not an admission that any or all of these matters form part of the prior art with respect to the disclosure.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, the recitation of “10-fold” includes 9-fold and 11-fold. As used herein, the use of a numerical range expressly includes all possible subranges, all individual numerical values within that range, including integers within such ranges and fractions of the values unless the context clearly indicates otherwise.

The present invention relates to compounds of Formula (I) or pharmaceutically acceptable salts thereof, corresponding pharmaceutical compositions, methods or processes of compound preparation, methods, compounds for use in, uses for and/or combination therapies for treating Nav1.8 mediated diseases, disorders, and conditions, such as pain and/or pain-associated disease(s), disorder(s) or condition(s), respectively, and atrial fibrillation.

The definitions for the various groups and substituent groups of any of the Formulas disclosed herein, or a pharmaceutically acceptable salt and/or a corresponding tautomer form thereof provided throughout the specification are intended to particularly describe each compound species disclosed herein, individually, as well as groups of one or more compound species.

As used herein, the term alkali metal is intended to mean the Group I elements, which include, but are not limited to lithium (Li), sodium (Na), or potassium (K) and the like. The term alkali earth metal may include, but are not limited to calcium (Ca) or magnesium (Mg) and the like.

As used herein, the terms “alkyl” or “straight or branched alkyl”, and the like, represent a saturated, straight or branched hydrocarbon moiety. Exemplary alkyls include, but are not limited to methyl (Me), ethyl (Et), propyl (e.g., n-propyl, isopropyl), butyl (e.g., n-butyl, isobutyl, tert-butyl), and pentyl (e.g., n-pentyl, isopentyl, neopentyl), etc. An alkyl group can have a specified number of carbon atoms. When a number appears in a subscript after the symbol “C,” the subscript defines with more specificity the number of carbon atoms which that particular alkyl can contain. For example, the terms “C₁-C₆” and “C₁₋₆” refer to an alkyl containing 1 to 6 carbon atoms and the terms “C₁-C₄” and “C₁₋₄” refer to an alkyl containing 1 to 4 carbon atoms.

When the term “alkyl” is used in combination with other substituent groups, such as “haloalkyl” or “hydroxyalkyl”, the term “alkyl” is intended to encompass a divalent saturated, straight or branched-chain hydrocarbon radical.

For example, the terms “haloalkyl” or “straight or branched haloalkyl” are intended to mean a saturated, straight or branched hydrocarbon moiety substituted with one or more halogens, where halogen is independently selected from: fluoro, chloro, bromo and iodo. A haloalkyl group can have a specified number of carbon atoms. For example, the terms “(C₁-C₆)haloalkyl” and “(C₁₋₆)haloalkyl” refer to a saturated, straight- or branched-chain haloalkyl radical, having at least 1 and up to 6 carbon atoms. Likewise, the terms “(C₁-C₄)haloalkyl” and “(C₁₋₄)haloalkyl” refer to a saturated, straight- or branched-chain haloalkyl radical having 1 to 4 carbon atoms. “Fluorinated alkyl” or “fluoroalkyl” in particular refers to any alkyl group as defined above substituted with at least one fluoro atom, e.g., one to three fluoro atoms, such as one, two, or three fluoroatoms. Representative haloalkyls include, but are not limited to trifluoromethyl (—CF₃), tetrafluoroethyl (—CF₂CHF₂), pentafluoroethyl (—CF₂CF₃) and the like.

The term “hydroxyalkyl” refers to a saturated, straight or branched hydrocarbon moiety substituted with one or more hydroxy groups.

As used herein, the terms “halogen” and “halo” mean fluoro (—F), chloro (—Cl), bromo (—Br), and iodo (—I).

“Hydroxy” or “hydroxyl” is intended to mean the radical —OH.

“Oxo” represents a double-bonded oxygen moiety; for example, if attached directly to a carbon atom forms a carbonyl moiety (C═O), or attached to an N or S forms oxides, e.g., N-oxides, sulfones or sulfoxides.

The term “cyano” refers to —CN.

The term “amino” refers to —NH₂. One or more hydrogen atoms of an amino group can be replaced by a substituent such as an alkyl group, which is referred to as an “alkylamino.” Alkylamino groups have one or both hydrogen atoms of an amino group replaced with an alkyl group and is attached to the parent molecule through a bond to the nitrogen atom of the alkylamino group. For example, alkylamino includes methylamino (—NHCH₃), dimethylamino (—N(CH₃)₂), —NHCH₂CH₃ and the like.

“Alkoxy” refers to a group containing an alkyl radical attached through an oxygen linking atom, wherein alkyl is as defined above. An alkoxy group can have a specified number of carbon atoms. For example, the terms “(C₁-C₆)alkoxy” and “(C₁₋₆)alkoxy” refer to an alkyl radical, having at least 1 and up to 6 carbon atoms attached through an oxygen linking atom. Likewise, the terms “(C₁-C₄)alkoxy” and “(C₁₋₄)alkoxy” refer to an alkyl radical having at least 1 and up to 4 carbon atoms attached through an oxygen linking atom. Exemplary alkoxy groups include, but are not limited to, methoxy, ethoxy, n-propoxy, isopropoxy, n-butoxy, s-butoxy, and t-butoxy.

“Haloalkoxy” refers to an alkoxy group in which the alkyl moiety is substituted with one or more halogens, wherein halogen is independently selected from fluoro, chloro, bromo, and iodo. A haloalkoxy group can have a specified number of carbon atoms. For example, the term “(C₁-C₆)haloalkoxy refers to a haloalkyl radical, having at 1 to 6 carbon atoms attached through an oxygen linking atom. Representative haloalkoxy groups include, but are not limited to difluoromethoxy (—OCHCF₂), trifluoromethoxy (—OCF₃), tetrafluoroethoxy (—OCF₂CHF₂) and the like.

In accordance with convention used in the art: ├ is used in structural formulas herein to depict the bond that is the point of attachment of a group, moiety or substituent to the core, backbone, or parent molecule structure.

When a bond to a substituent is shown to cross a bond connecting two atoms in a ring, then such substituent can be bonded to any atom on the ring.

As used herein, the term “compound(s) of the invention” means a compound of any of the Formulas disclosed herein, in any form, i.e., any salt or non-salt form (e.g., as a free acid or base form, or as a pharmaceutically acceptable salt thereof), any tautomer form thereof, and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” means that a group (e.g., alkyl, etc.), may be unsubstituted, or the group may be substituted with one or more specified substituent(s) as defined herein throughout the instant specification. The term “substituted” as used herein with respect to a group (e.g., alkyl, etc.) means that at least one hydrogen atom is replaced with a non-hydrogen group, provided that all normal valencies are maintained and that the substitution results in a stable compound. In the case where groups may be selected from a number of alternative groups the selected groups may be the same or different. For example, various substituent groups of compound formulas as defined in the present invention may be optionally substituted, but are not limited to substituents, such as halo, cyano, amino, alkyl, haloalkyl, alkoxy, and the like.

The term “independently” when used with reference to a substituent or heteroatom means that where more than one substituent or heteroatom is selected from a number of possible substituents or heteroatoms, respectively, those substituents or heteroatoms may be the same or different.

### Compounds

Compounds of formula (I) of the present invention are prodrugs of their respective parent compounds, which are Nav1.8 inhibitor compounds. Upon administration of the prodrug, the prodrug moiety is cleaved thereby resulting in the parent compound. Accordingly, Nav1.8 inhibitory activity upon administration of the prodrug is primarily due to formation of the parent compound from cleavage of the prodrug.

The prodrugs of the present invention typically have higher aqueous solubility than the corresponding parent compounds. This higher solubility facilitates administration of higher doses of the prodrug, resulting in a greater drug load per unit dosage. Thus, the compounds of formula (I) of the invention (i.e., prodrugs) may be advantageous for intravenous (IV) formulation and administration, and thus beneficial for use in the treatment of pain and pain associated diseases, disorders, and conditions in which administration of higher doses or administration via the IV route may be beneficial, such as treatment of acute pain.

The term “prodrug” refers to compounds that are drug precursors which, following administration and/or absorption, release the parent compound in vivo via a metabolic process. Typically, a prodrug has less biological activity than the parent compound. A prodrug may also improve the physical properties and/or efficacy of the parent compound, such as reduced toxicity and fewer unwanted effects through greater control of the absorption, blood levels, metabolic distribution and/or cellular uptake of the parent compound. Prodrugs may also have higher solubility than the corresponding parent compound.

The terms “parent compound” and “parent drug” refer to the biologically active entity that is released via enzymatic action of a metabolic or catabolic process, or via a chemical process following administration of the prodrug. The parent compound may also be the starting material for the preparation of the corresponding prodrug.

In one aspect, the present invention relates to a compound of Formula (I):

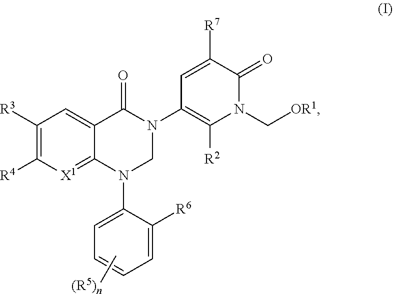

or a pharmaceutically acceptable salt thereof,
wherein:
   X¹ is N or CH;
   R¹ is —PO(OH)₂;
   R² is hydrogen, -(C₁₋₆)alkyl, —NR^{a}R^{b}, halo, or -(C₁₋₆)haloalkyl;
   each of R³ and R⁴ is independently hydrogen, halo, cyano, —NR^{a}R^{b}, -(C₁₋₆)alkyl, -(C₁₋₆)haloalkyl, —O—(C₁₋₆)alkyl, or —O—(C₁₋₆)haloalkyl;
   each of R⁵ is independently halo, -(C₁₋₆)alkyl, —O(C₁₋₆)alkyl, or —O(C₁₋₆)haloalkyl;
   R⁶ is hydrogen or -(C₁₋₆)alkyl;
   R⁷ is hydrogen, -(C₁₋₆)alkyl, halo, or -(C₁₋₆)haloalkyl;
   each of R^{a} and R^{b} is independently hydrogen or -(C₁₋₆)alkyl; and
   n is 0, 1, or 2.

In an embodiment of a compound of formula (I), or a pharmaceutically acceptable salt thereof, X¹ is N.

In an embodiment of a compound of formula (I), or a pharmaceutically acceptable salt thereof, X¹ is CH.

In an embodiment of a compound of formula (I), or a pharmaceutically acceptable salt thereof, R² is hydrogen.

In an embodiment of a compound of formula (I), or a pharmaceutically acceptable salt thereof, R² is -(C₁₋₆)alkyl.

In an embodiment of a compound of formula (I), or a pharmaceutically acceptable salt thereof, R² is CH₃.

In an embodiment of a compound of formula (I), or a pharmaceutically acceptable salt thereof, R² is —NR^{a}R^{b}.

In an embodiment of a compound of formula (I), or a pharmaceutically acceptable salt thereof, R² is —NH₂.

In an embodiment of a compound of formula (I), or a pharmaceutically acceptable salt thereof, R³ is hydrogen, halo, or -(C₁₋₆)haloalkyl.

In an embodiment of a compound of formula (I), or a pharmaceutically acceptable salt thereof, R³ is hydrogen, —Cl, or —CF₃.

In an embodiment of a compound of formula (I), or a pharmaceutically acceptable salt thereof, R³ is hydrogen.

In an embodiment of a compound of formula (I), or a pharmaceutically acceptable salt thereof, R³ is —Cl.

In an embodiment of a compound of formula (I), or a pharmaceutically acceptable salt thereof, R³ is —CF₃.

In an embodiment of a compound of formula (I), or a pharmaceutically acceptable salt thereof, R⁴ is hydrogen, halo, or -(C₁₋₆)haloalkyl.

In an embodiment of a compound of formula (I), or a pharmaceutically acceptable salt thereof, R⁴ is hydrogen, —Cl, or —CF₃.

In an embodiment of a compound of formula (I), or a pharmaceutically acceptable salt thereof, R⁴ is hydrogen.

In an embodiment of a compound of formula (I), or a pharmaceutically acceptable salt thereof, R⁴ is —Cl.

In an embodiment of a compound of formula (I), or a pharmaceutically acceptable salt thereof, R⁴ is —CF₃.

In an embodiment of a compound of formula (I), or a pharmaceutically acceptable salt thereof, one of R³ and R⁴ is hydrogen and the other of R³ and R⁴ is halo, cyano, —NR^{a}R^{b}, -(C₁₋₆)alkyl, -(C₁₋₆)haloalkyl, —O—(C₁₋₆)alkyl, or —O—(C₁₋₆)haloalkyl.

In an embodiment of a compound of formula (I), or a pharmaceutically acceptable salt thereof, one of R³ and R⁴ is hydrogen and the other of R³ and R⁴ is halo or -(C₁. 6)haloalkyl.

In an embodiment of a compound of formula (I), or a pharmaceutically acceptable salt thereof, one of R³ and R⁴ is hydrogen and the other of R³ and R⁴ is —Cl or —CF₃.

In an embodiment of a compound of formula (I), or a pharmaceutically acceptable salt thereof, each R⁵ is independently halo or —O(C₁₋₆)haloalkyl.

In an embodiment of a compound of formula (I), or a pharmaceutically acceptable salt thereof, each R⁵ is independently —F or —OCF₃.

In an embodiment of a compound of formula (I), or a pharmaceutically acceptable salt thereof, n is 1 and R⁵ is —F.

In an embodiment of a compound of formula (I), or a pharmaceutically acceptable salt thereof, n is 1 and R⁵ is —OCF₃.

In an embodiment of a compound of formula I or a pharmaceutically acceptable salt thereof,

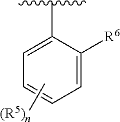

has the structure:

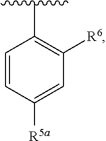

wherein R^{5a} is halo, -(C₁₋₆)alkyl, —O(C₁₋₆)alkyl, or —O(C₁₋₆)haloalkyl

In an embodiment of a compound of formula (I), or a pharmaceutically acceptable salt thereof, R^{5a} is halo or —O(C₁₋₆)haloalkyl.

In an embodiment of a compound of formula (I), or a pharmaceutically acceptable salt thereof, R^{5a} is —F or —OCF₃.

In an embodiment of a compound of formula (I), or a pharmaceutically acceptable salt thereof, R^{5a} is —F.

In an embodiment of a compound of formula (I), or a pharmaceutically acceptable salt thereof, R^{5a} is —OCF₃.

In an embodiment of a compound of formula (I), or a pharmaceutically acceptable salt thereof,

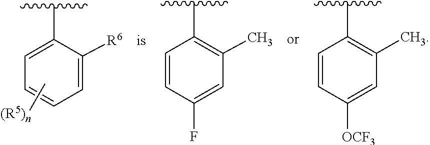

In an embodiment of a compound of formula (I), or a pharmaceutically acceptable salt thereof, R⁶ is hydrogen.

In an embodiment of a compound of formula (I), or a pharmaceutically acceptable salt thereof, R⁶ is -(C₁₋₆)alkyl.

In an embodiment of a compound of formula (I), or a pharmaceutically acceptable salt thereof, R⁶ is —CH₃, —CH₂CH₃, or —CH(CH₃)₂.

In an embodiment of a compound of formula (I), or a pharmaceutically acceptable salt thereof, R⁶ is —CH₃.

In an embodiment of a compound of formula (I), or a pharmaceutically acceptable salt thereof, R⁷ is hydrogen.

In an embodiment of a compound of formula (I), or a pharmaceutically acceptable salt thereof, R⁷ is -(C₁₋₆)alkyl.

In an embodiment of a compound of formula (I), or a pharmaceutically acceptable salt thereof, R⁷ is halo.

In an embodiment of a compound of formula (I), or a pharmaceutically acceptable salt thereof, R⁷ is —F or —Cl.

In an embodiment of a compound of formula (I), or a pharmaceutically acceptable salt thereof,
X¹ is N;
R¹ is —PO(OH)₂;
R² is hydrogen or -(C₁₋₆)alkyl;
one of R³ and R⁴ is hydrogen and the other of R³ and R⁴ is halo or -(C₁₋₆)haloalkyl;
R⁵ is halo or —O(C₁₋₆)haloalkyl;
R⁶ is -(C₁₋₆)alkyl;
R⁷ is hydrogen; and
n is 1.

In an embodiment of a compound of formula (I), or a pharmaceutically acceptable salt thereof,
X¹ is N;
R¹ is —PO(OH)₂;
R² is —CH₃;
one of R³ and R⁴ is hydrogen and the other of R³ and R⁴ is halo or -(C₁₋₆)haloalkyl;
R⁵ is halo or —O(C₁₋₆)haloalkyl;
R⁶ is -(C₁₋₆)alkyl;
R⁷ is hydrogen; and
n is 1.

In an embodiment of a compound of formula (I), or a pharmaceutically acceptable salt thereof,
X¹ is N;
R¹ is —PO(OH)₂;
R² is —CH₃;
one of R³ and R⁴ is hydrogen and the other of R³ and R⁴ is —Cl or —CF₃;
R⁵ is —F or —OCF₃;
R⁶ is —CH₃;
R⁷ is hydrogen; and
n is 1.

In an embodiment of a compound of formula (I), or a pharmaceutically acceptable salt thereof,
X¹ is C;
R¹ is —PO(OH)₂;
R² is hydrogen or -(C₁₋₆)alkyl;
one of R³ and R⁴ is hydrogen and the other of R³ and R⁴ is halo or -(C₁₋₆)haloalkyl;
R⁵ is halo or —O(C₁₋₆)haloalkyl;
R⁶ is -(C₁₋₆)alkyl;
R⁷ is hydrogen; and
n is 1.

In an embodiment of a compound of formula (I), or a pharmaceutically acceptable salt thereof,
X¹ is C;
R¹ is —PO(OH)₂;
R² is —CH₃;
one of R³ and R⁴ is hydrogen and the other of R³ and R⁴ is halo or -(C₁₋₆)haloalkyl;
R⁵ is halo or —O(C₁₋₆)haloalkyl;
R⁶ is -(C₁₋₆)alkyl;
R⁷ is hydrogen; and
n is 1.

In an embodiment of a compound of formula (I), or a pharmaceutically acceptable salt thereof,
X¹ is C;
R¹ is —PO(OH)₂;
R² is —OH₃;
one of R³ and R⁴ is hydrogen and the other of R³ and R⁴ is —Cl or —OF₃;
R⁵ is —F or1-nF₃;
R⁶ is —OH₃;
R⁷ is hydrogen; and
n is 1.

In another aspect, the invention relates to a compound which is selected from:

| Name | Structure |
|---|---|
| (5-(1-(4-Fluoro-2-methylphenyl)-4-oxo-6- (trifluoromethyl)-1,4-dihydroquinazolin- 3(2H)-yl)-6-methyl-2-oxopyridin-1(2H)- yl)methyl dihydrogen phosphate | 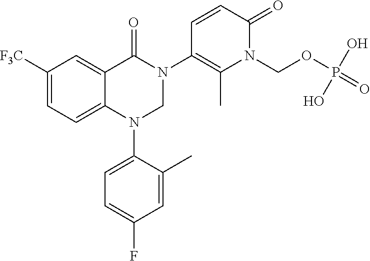 [SMILES: Cc1cc(F)ccc1N1CN(c2ccc(=O)n(COP(=O)(O)O)c2C)C(=O)c2cc(C(F)(F)F)ccc21] |
| (5-(1-(4-fluoro-2-methylphenyl)-4-oxo-7- (trifluoromethyl)-1,4-dihydroquinazolin- 3(2H)-yl)-6-methyl-2-oxopyridin-1(2H)- yl)methyl dihydrogen phosphate | 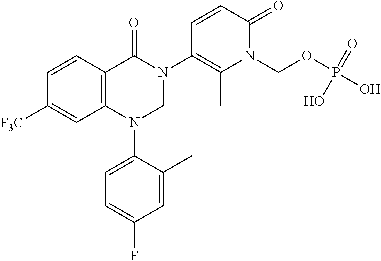 [SMILES: Cc1cc(F)ccc1N1CN(c2ccc(=O)n(COP(=O)(O)O)c2C)C(=O)c2ccc(C(F)(F)F)cc21] |
| (5-(6-chloro-1-(4-fluoro-2-methylphenyl)-4- oxo-1,4-dihydroquinazolin-3(2H)-yl)-6- methyl-2-oxopyridin-1(2H)-yl)methyl dihydrogen phosphate | 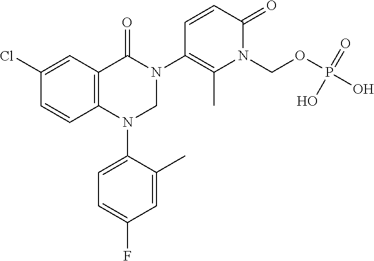 [SMILES: Cc1cc(F)ccc1N1CN(c2ccc(=O)n(COP(=O)(O)O)c2C)C(=O)c2cc(Cl)ccc21] |
| (6-methyl-5-(1-(2-methyl-4- (trifluoromethoxy)phenyl)-4-oxo-6- (trifluoromethyl)-1,4-dihydropyrido[2,3- d]pyrimidin-3(2H)-yl)-2-oxopyridin-1(2H)- yl)methyl dihydrogen phosphate | 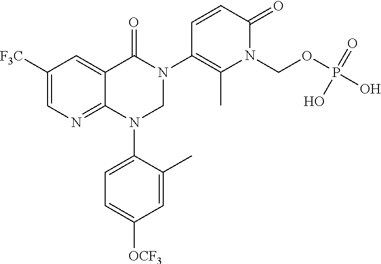 [SMILES: Cc1cc(OC(F)(F)F)ccc1N1CN(c2ccc(=O)n(COP(=O)(O)O)c2C)C(=O)c2cc(C(F)(F)F)cnc21] |

or a pharmaceutically acceptable salt thereof.

### Salts

Because of their potential use in medicine, the salts of the compounds of any of the Formulas disclosed herein, including Formula (I) are preferably pharmaceutically acceptable salts. Pharmaceutically acceptable salts include, among others, those described by Berge, Bighley and Monkhouse J.Pharm.Sci (1977) 66, pp 1-19, or those listed in PH Stahl and CG Wermuth, editors, Handbook of Pharmaceutical Salts; Properties, Selection and Use, Second Edition Stahl/Wermuth: Wiley-VCH/VHCA, 2011. Non-pharmaceutically acceptable salts may be used, for example as intermediates in the preparation of a compound of any of the Formulas disclosed herein or a pharmaceutically acceptable salt thereof.

Suitable pharmaceutically acceptable salts can include acid or base addition salts. Such base additional salts can be formed by reaction of a compound of any of the Formulas disclosed herein, including Formula (I) of the invention with the appropriate base, optionally in a suitable solvent such as an organic solvent, to give the salt which can be isolated by a variety of methods, including crystallisation and filtration.

Such acid addition salts can be formed by reaction of a compound of any of the Formulas disclosed herein, including Formula (I) of the invention, with the appropriate acid, optionally in a suitable solvent such as an organic solvent, to give the salt which can be isolated by a variety of methods, including crystallisation and filtration.

Salts may be prepared in situ during the final isolation and purification of a compound of any of the Formulas disclosed herein, including Formula (I) of the invention. If a basic compound of any of the Formulas disclosed herein, including Formula (I) of the invention, is isolated as a salt, the corresponding free base form of that compound may be prepared by any suitable method known to the art, including treatment of the salt with an inorganic or organic base. Similarly, if a compound of any of the Formulas disclosed herein, including Formula (I) of the invention, containing an acidic functional group such as a phosphate group is isolated as a salt, the corresponding free acid form of that compound may be prepared by any suitable method known to the art, including treatment of the salt with an inorganic or organic acid.

For example, when a compound of the invention is a base (contain a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like.

If an inventive basic compound is isolated as a salt, the corresponding free base form of that compound may be prepared by any suitable method known to the art, including treatment of the salt with an inorganic or organic base, suitably an inorganic or organic base having a higher pKa than the free base form of the compound.

When a compound of the invention is an acid (contains an acidic moiety), a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Certain of the compounds of this invention may form salts with one or more equivalents of an acid (if the compound contains a basic moiety) or a base (if the compound contains an acidic moiety). The present invention includes within its scope all possible stoichiometric and non-stoichiometric salt forms. It will be understood that if a compound of any of the Formulas disclosed herein, including Formula (I) as defined herein contains two or more basic moieties, the stoichiometry of salt formation may include 1, 2 or more equivalents of acid. Such salts would contain 1, 2 or more acid counterions, for example, a dihydrochloride salt. Stoichiometric and non-stoichiometric forms of a pharmaceutically acceptable salt of a compound of any of the Formulas disclosed herein, including Formula (I) of the invention are included within the scope of the invention, including sub-stoichiometric salts, for example where a counterion contains more than one acidic proton.

Because the compounds of this invention may contain both acid and base moieties, pharmaceutically acceptable salts may be prepared by treating these compounds with an alkaline reagent or an acid reagent, respectively. Accordingly, this invention also provides for the conversion of one pharmaceutically acceptable salt of a compound of this invention, e.g., a hydrochloride salt, into another pharmaceutically acceptable salt of a compound of this invention, e.g., a sodium salt.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

In particular embodiments, the invention provides a pharmaceutically acceptable salt of of a compound of formula (I). For example, the phosphate group of R¹ (—PO(OH)₂) is an acidic moiety that is particularly likely to participate in salt formation with cationic species (i.e., base addition salts). Any of the base addition salts listed above can be used to form a pharmaceutically acceptable salt of a compound of formula (I) of the invention. Other functional groups of a compound of formula (I) may additionally or alternatively participate in salt formation with acid and/or base addition salts, such as those described above.

In an embodiment of a pharmaceutically acceptable salt of a compound of formula (I), R¹ is —P(O)(OH)O⁻M⁺, —PO(O⁻)₂·2M⁺, or —PO(O⁻)₂·D²⁺;
each M⁺ is independently a pharmaceutically acceptable monovalent cation; and
D²⁺ is a pharmaceutically acceptable divalent cation.

In some embodiments, monovalent cations (M⁺) suitable for use in the invention include, but are not limited to, alkali metal ions, e.g., lithium (Li⁺), sodium (Na⁺), potassium (K⁺), etc.; ammonium ions (e.g., —N(R^{a})₄, wherein each R^{a} is independently hydrogen, cyclohexyl, or -(C₁₋₆)alkyl), the -(C₁₋₆)alkyl being optionally substituted with one or more, suitably 1-6, —OH groups), such as NH₄⁺, ethanolamine ion

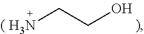

N-methyl-D-glucamine ion, dicyclohexylamine ion, etc. When two M⁺ are present, each M⁺ is independently a monovalent cation, wherein each M⁺ is the same or different, preferably each M⁺ is the same.

In an embodiment, each M⁺ is independently an alkali metal ion.

In an embodiment, each M⁺ is independently Li⁺, Na⁺, or K⁺.

In an embodiment, each M⁺ is independently NH₄⁺.

In an embodiment, each M⁺ is independently

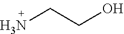

(ethanolamine ion).

In some embodiments, divalent cations (D²⁺) suitable for use in the invention include, but are not limited to, alkaline earth metal ions, e.g., magnesium (Mg²⁺), calcium (Ca²⁺), strontium (Sr²⁺), etc.; divalent aluminum ions; etc.

In an embodiment, D²⁺ is alkaline earth metal ion.

In an embodiment, D²⁺ is Mg²⁺ or Ca²⁺.

Other monovalent and divalent cations suitable for use in the invention include monovalent or divalent ions of amino acid ions, such as monovalent or divalent ions arginine, lysine, ornithine, etc. Monovalent and divalent cations including basic nitrogen-containing groups can be prepared by quaternization with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), etc.

### Enantiomers, Diastereomers, and Polymorphs

The compounds according to any of the Formulas disclosed herein, including Formula (I), or a pharmaceutically acceptable salt thereof of the invention, may contain one or more asymmetric center(s) (i.e., also referred to as a chiral center) and may, therefore, exist in optically forms (e.g., as individual enantiomers, diastereomers, or other stereoisomeric forms, or as mixtures thereof) and racemic forms. All of these individual compounds, stereoisomers, and mixtures thereof are included within the scope of the invention.

Chiral centers, such as chiral carbon atoms, may also be present in a substituent such as an alkyl group. Where the stereochemistry of a chiral center present in any of the Formulas disclosed herein, including Formula (I), or a pharmaceutically acceptable salt thereof of the invention, or in any chemical structure illustrated herein, is not specified the structure is intended to encompass all individual stereoisomers and all mixtures thereof. Thus, compounds or a pharmaceutically acceptable salt thereof of the invention containing one or more chiral centers may be used as racemic mixtures, enantiomerically enriched mixtures, or as enantiomerically pure individual stereoisomers.

Individual stereoisomers of a compound according to any of the Formulas disclosed herein, including Formula (I), or a pharmaceutically acceptable salt thereof of the invention, which contain one or more asymmetric centers may be resolved by methods known to those skilled in the art. For example, such resolution may be carried out:
(1) by formation of diastereoisomeric salts, complexes or other derivatives;
(2) by selective reaction with a stereoisomer-specific reagent, for example by enzymatic oxidation or reduction; or
(3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form.

Alternatively, specific stereoisomers may be synthesized by asymmetric synthesis using optically active reagents, substrates, catalysts or solvents, or by converting one enantiomer to the other by asymmetric transformation.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.”

It is to be understood that when named or depicted by structure, the disclosed compound, or solvates (particularly, hydrates) thereof, also include all polymorphs thereof.

Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

### Solvates

Compounds of the invention, or pharmaceutically acceptable salts thereof may exist in solvated and unsolvated forms. For solvates of the compounds of the invention, or pharmaceutically acceptable salts thereof, that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water.

### Deuterated Compounds

The invention also includes various deuterated forms of the compounds of any of the Formulas disclosed herein, including Formula (I) or a pharmaceutically acceptable salt thereof of the invention. Each available hydrogen atom attached to a carbon atom may be independently replaced with a deuterium atom.

A person of ordinary skill in the art will know how to synthesize deuterated forms of the compounds of any of the Formulas disclosed herein, including Formula (I) or a pharmaceutically acceptable salt thereof of the invention. For example, deuterated materials, such as alkyl groups may be prepared by conventional techniques (see for example: methyl-d₃-amine available from Aldrich Chemical Co., Milwaukee, WI, Cat. No.489,689-2).

### Isotopes

The invention also includes isotopically-labeled compounds which are identical to those recited in any of the Formulas disclosed herein, including Formula (I) or a pharmaceutically acceptable salt thereof of the invention but for the fact that one or more atoms are replaced by an atom having an atomic mass or mass number different from the atomic mass or mass number most commonly found in nature.

Examples of isotopes that can be incorporated into compounds of the invention include isotopes of hydrogen, carbon, nitrogen, oxygen, fluorine, iodine and chlorine such as ³H, ¹¹C, ¹⁴C ¹⁸F, ¹²³I or ¹²⁵I.

Compounds of the invention and pharmaceutically acceptable salts of said compounds that contain the aforementioned isotopes and/or other isotopes of other atoms are within the scope of the invention. Isotopically labeled compounds of the invention, for example those into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

### Purity

Because the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing more pure forms used in the pharmaceutical compositions.

It is recognized that the compounds of any of the Formulas disclosed herein, including Formula (I) or a pharmaceutically acceptable salt thereof of the invention may exist in forms as stereoisomers, regioisomers, or diastereoisomers.

### Tautomers

Moreover, compounds of the invention may exist as tautomers or in tautomeric forms. It is conventionally understood in the chemical arts that tautomers are structural or constitutional isomers of chemical compounds that readily interconvert. This reaction commonly results in the relocation of a proton. A structural isomer, or constitutional isomer (per IUPAC), is a type of isomer in which molecules with the same molecular formula have different bonding patterns and atomic organization, as opposed to stereoisomers, in which molecular bonds are always in the same order and only spatial arrangement differs. The concept of tautomerizations is called tautomerism. The chemical reaction interconverting the two is called tautomerization. Care should be taken not to confuse tautomers with depictions of ‘contributing structures’ in chemical resonance. Tautomers are distinct chemical species and can be identified as such by their differing spectroscopic data, whereas resonance structures are merely convenient depictions and do not physically exist.

### Synthetic Schemes and General Methods of Preparation

The present invention also relates to processes for making compounds of any of the Formulas disclosed herein, including Formula (I) or a pharmaceutically acceptable salt thereof of the invention.

The compounds of any of the Formulas disclosed herein, or a pharmaceutically acceptable salt thereof of the invention may be made by any number of processes using conventional organic syntheses as described in the Schemes below and more specifically illustrated by the exemplary compounds which follow in the Examples section herein, or by drawing on the knowledge of a skilled organic chemist. Suitable synthetic routes are depicted below in the following general reaction schemes.

The synthesis provided in these Schemes are applicable for producing compounds of the invention as defined by any of the Formulas disclosed herein, having a variety of different functional groups as defined employing appropriate precursors, which are suitably protected if needed, to achieve compatibility with the reactions outlined herein. Subsequent deprotection, where needed, affords compounds of the nature generally disclosed. While the Schemes are shown with compounds only as defined therein, they are illustrative of processes that may be used to make the compounds of the invention.

Intermediates (compounds used in the preparation of the compounds of the invention) also may be present as salts. Thus, in reference to intermediates, the phrase “compound(s) of formula (number)” means a compound having that structural formula or a pharmaceutically acceptable salt thereof.

The compounds of the invention may be obtained by using the procedures illustrated in the Schemes below, or by applying appropriate synthetic organic chemistry procedures and methodology known to those of skill in the art.

The methods provided in these Schemes can be used to prepare compounds of the invention containing a variety of different X¹, R¹, R², R³, R⁴, R⁵, and R⁶ groups (descriptions shown above for compounds of Formula (I)) employing appropriate precursors.

Those skilled in the art will appreciate that in the preparation of compounds of the invention (e.g., compounds of Formula (I) or a pharmaceutically acceptable salt thereof), it may be necessary and/or desirable to protect one or more sensitive groups in the molecule or the appropriate intermediate to prevent undesirable side reactions. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups for use according to the present invention are well-known to those skilled in the art and may be used in a conventional manner. See for example, “Protective Groups in Organic Synthesis” by T. W. Green and P. G. M Wets (Wiley & Sons, 1991) or “Protecting Groups” by P. J. Kocienski (Georg Thieme Verlag, 1994). Subsequent deprotection, where needed, affords compounds of the nature generally disclosed.

In some instances, a substituent may be specifically selected to be reactive under the reaction conditions used. Under these circumstances, the reaction conditions convert the selected substituent into another substituent that is either useful as an intermediate compound or is a desired substituent in a target compound.

While the Schemes shown below are representative of methods for preparing compounds of Formula (I), they are only intended to be illustrative of processes that may be used to make the compounds of the invention.

Compound names were generated using the software naming program ChemDraw Ultra v12.0, available from Perkin Elmer, 940 Winter Street, Waltham, Massachusetts, 02451, USA. (http://www.perkinelmer.com/).

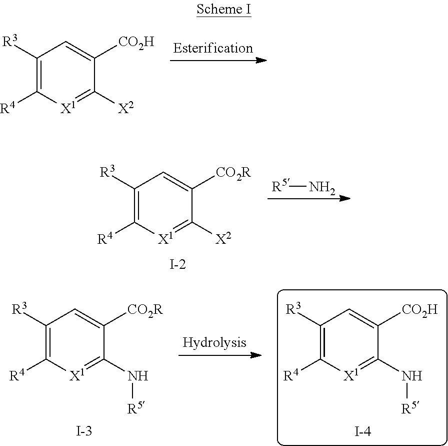

The preparation of the compounds of the present invention typically begins with the synthesis of N-substituted-2-aminoaromatic acid derivatives I-4 (Scheme I). Esterification of a suitably substituted 2-halo aromatic acid under standard conditions provides the corresponding ester I-2. Typically, esterification reactions are performed under either acidic conditions, in the presence of an alcohol, or under basic conditions, in the presence of a suitable alkyl halide. Reaction of the 2-halo aromatic ester I-2 (X²=Cl, Br or I) with an appropriate aniline or amine (R^{5′}—NH₂; R^{5′} is a substituted phenyl group) provides the corresponding N-substituted-2-aminoaromatic esters I-3. Typically, this reaction is performed at elevated temperature, using either standard heating or microwave irradiation, in the presence of a catalyst, for example Pd₂(dba)₃ or Cu/CuO, a suitable ligand, for instance BINAP or Xantphos, and an inorganic base, typically Cs₂CO₃ or K₂CO₃, in an appropriate solvent, such as 1,4-dioxane, toluene or 2-ethoxyethanol.

Saponification of the ester 1-3 to the corresponding N-substituted-2-aminoaromatic acid derivatives 1-4 is typically achieved under standard basic conditions, using bases such as LiOH, KOH, or NaOH, in a suitable solvent or solvent system, for instance methanol/H₂O, ethanol/H₂O, or THF/H₂O. Such conditions are well-known to those of skill in the art.

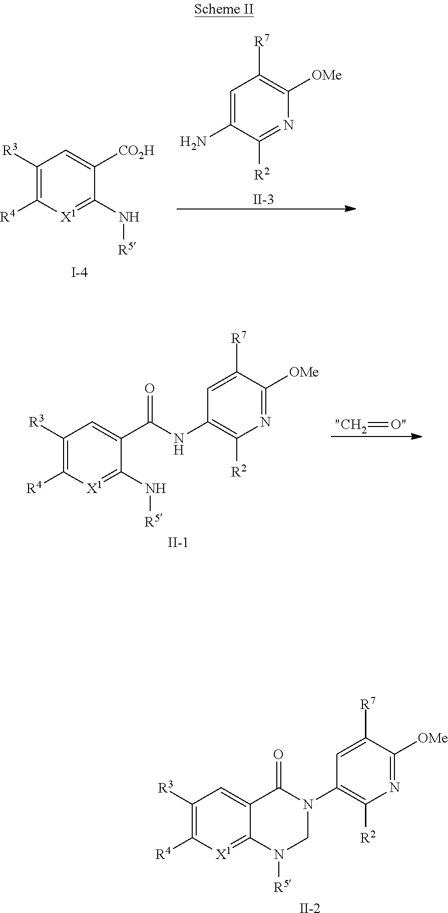

The intermediate N-substituted-2-aminoaromatic acid derivatives 1-4, prepared as illustrated in Scheme I, can be converted to compound II-2 as outlined in Scheme II. Coupling of 1-4 with a substituted methoxy pyridyl amine II-3 under various amide coupling conditions known to those of skill in the art, provides the corresponding amide I-1. For example, one might employ standard coupling reagents, like EDC/HOBT, HATU, HBTU or T3P, in the presence of an amine base, like triethylamine, or Hunig's base (diisopropylethylamine), in a suitable solvent, typically DMF, DMA or acetonitrile. Alternatively, one might convert the acid to the corresponding acid chloride, using a reagent such as thionyl chloride or oxalyl chloride, and the like, then react the acid chloride with a substituted methoxy pyridyl amine II-3, in the presence of an acid scavenger or base, such as pyridine, 2,6-lutidine, triethylamine or Hunig's base, in an appropriate solvent, such as dichloromethane or pyridine, to afford the desired coupling product I-1.

Formation of the dihydroquinazolinone ring system, as in intermediate II-2, involves reaction of intermediate II-1 with formaldehyde or a suitable equivalent. For instance, the reaction may be achieved using formaldehyde, either as gaseous formaldehyde, paraformaldehyde, or s-trioxane, in the presence of an acid, preferably PTSA or sulfuric acid. Alternatively, the dihydroquinazolinone ring system can be formed via reaction of I-1 using diiodomethane or chloroiodomethane as a formaldehyde equivalent. In this variant of the cyclization reaction, a base, typically Cs₂CO₃ or NaH, is used, in a suitable solvent, oftentimes acetonitrile or DMF. The choice of using formaldehyde or diiodomethane depends on the particular reactivity characteristics of the intermediate I-1.

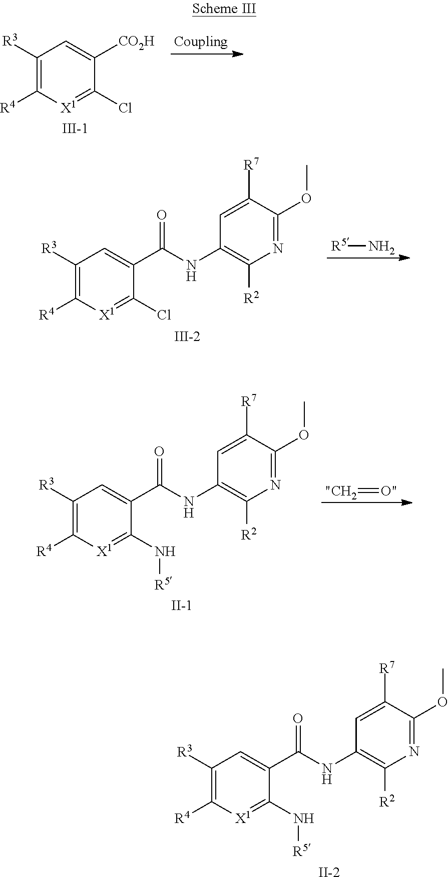

In a variation of the methods described in Schemes I and II, the compounds of the present invention can be prepared as illustrated in Scheme III. Coupling of intermediate III-1 (typically obtained from commercially available sources) with a substituted methoxy pyridyl amine II-3 under various amide coupling conditions known to those of skill in the art, provides the corresponding amide III-2. General conditions for forming amides are described in Scheme II. Subsequently, amide III-2 can be reacted with an appropriate aniline or amine (R^{5′}—NH₂) under similar conditions as described for conversion of I-2 to 1-3 in Scheme I to afford intermediate I-1. Intermediate I-1 can then be converted to compound II-2 according to the methods illustrated in Scheme II.

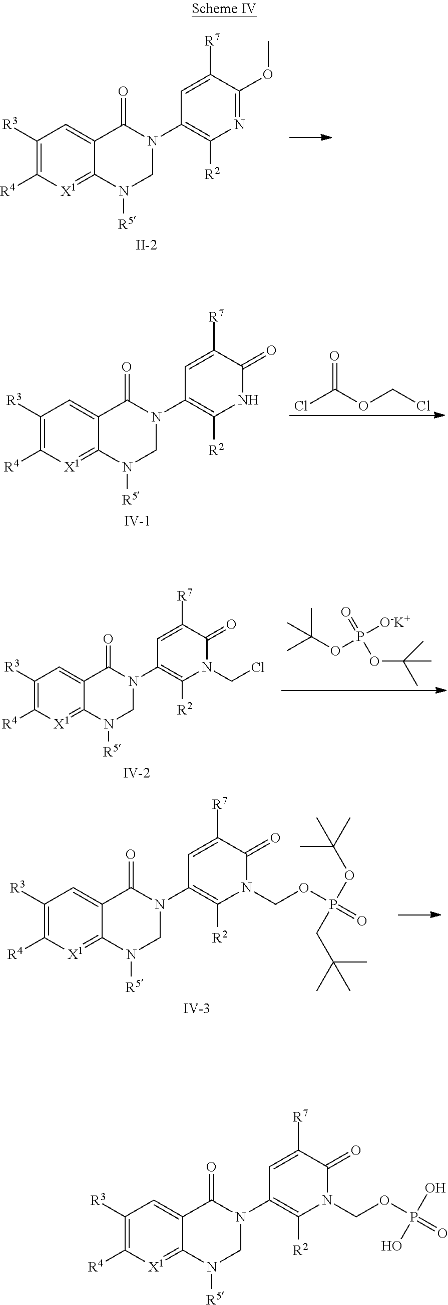

As shown in Scheme IV, transformation of compound II-2 to the pyridone IV-1 can be achieved by reacting compound II-2 with a mixture of TMS-chloride and NaI, or a solution of TMS-iodide, in a neutral solvent like acetonitrile, at elevated temperature. Compound IV-1 can be reacted with chloromethyl chloroformate in the presence of an organic base such as DABCO in suitable solvents such as EtOAc and DMF to provide the chloromethylpyridone IV-2. Reaction of compound IV-2 with potassium di-tert-butyl phosphate in the presence of a phase transfer catalyst such as TBAI in solvent DMF at elevated temperature provides compound IV-3. Removal of the tert-butyl protecting groups under acidic conditions such as acetic acid in acetonitrile and water provides the prodrug compounds of the invention.

### Pharmaceutical Compositions, Administration Routes, and Dosages

The compounds of the invention may be formulated into pharmaceutical compositions prior to administration to a subject. According to one aspect, the invention provides a pharmaceutical composition comprising a compound of the invention (i.e. a compound as defined by any of the Formulas disclosed herein, including Formula (I) or a pharmaceutically acceptable salt thereof of the invention) and one or more pharmaceutically acceptable excipients. According to one aspect, the invention provides a pharmaceutical composition comprising a compound of the invention (i.e. a compound as defined by any of the Formulas disclosed herein, including Formula (I) or a pharmaceutically acceptable salt thereof) and a pharmaceutically acceptable excipient.

In another aspect, the invention relates to a pharmaceutical composition or formulation, which comprises: a compound of formula (I), or a pharmaceutically acceptable salt thereof of the invention; a pharmaceutically acceptable excipient(s); and optionally one or more other therapeutic ingredients.

The pharmaceutical compositions or formulations as defined herein typically contain one compound of the invention. However, in certain embodiments, the pharmaceutical compositions may contain more than one compound of the invention. In addition, the pharmaceutical compositions of the invention may optionally further comprise one or more additional pharmaceutically active compounds.

A pharmaceutically acceptable excipient is non-toxic and should not interfere with the efficacy of the active ingredient. Suitable pharmaceutically acceptable excipients will vary depending upon the particular dosage form chosen, route of administration, etc. Suitable pharmaceutically acceptable excipients include the following types of excipients: diluents, carriers, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. Examples of pharmaceutically acceptable excipients are described, e.g., in Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

Pharmaceutical compositions may be adapted for administration by any appropriate or suitable route, for example by systemic administration (e.g., oral administration, parenteral administration, transdermal administration, rectal administration, inhalation), topical administration, etc. Parenteral administration is typically by injection or infusion and includes intravenous, intramuscular, and subcutaneous injection or infusion. Inhalation refers to administration into the patient's lungs whether inhaled through the mouth or through the nasal passages. Typically, administration is via the oral route or parenteral route.

Pharmaceutical compositions adapted for oral administration may be presented as solid dosage forms such as tablets, capsules, caplets, troches, pills; powders; or liquid dosage forms such as solutions, suspensions, syrups, elixirs, or emulsion, etc. Pharmaceutical compositions adapted for parenteral administration (e.g., intravenous administration) may be presented as solutions, suspensions, and powders for reconstitution.

In general, pharmaceutical compositions of the invention are prepared using conventional materials and techniques, such as mixing, blending and the like. Some of the methods commonly used in the art are described in Remington's Pharmaceutical Sciences (Mack Publishing Company).

Solid oral dosage forms, such as tablets and capsules can be prepared by mixing a compound of the invention with excipients such as diluents and fillers (e.g., starch, lactose, sucrose, calcium carbonate, calcium phosphate and the like), binders (e.g., starch, acacia gum, carboxymethyl cellulose, hydroxypropyl cellulose, crystalline cellulose, and the like), lubricants (e.g., magnesium stearate, talc and the like), and the like.

Pharmaceutical compositions adapted for parenteral administration can be an injection solution prepared from powders, granules or tablets by mixing with a carrier, such as distilled water, saline and the like, and base and the like may be used for pH adjustment.

In one embodiment, a pharmaceutical composition of the invention is formulated for oral administration.

In another embodiment, a pharmaceutical composition of the invention is formulated for parenteral administration, particularly intravenous administration.

The invention also provides a pharmaceutical composition comprising from 0.5 to 1,000 mg of a compound of the invention (i.e., a compound of any of the Formulas disclosed herein, including Formula (I) or a pharmaceutically acceptable salt thereof of the invention) and from 0.5 to 1,000 mg of a pharmaceutically acceptable excipient.

Compounds and pharmaceutical compositions of the invention as defined herein may be administered once or according to a dosing regimen, where a number of doses are administered at varying intervals of time for a given period of time. For example, doses may be administered one, two, three, or four times per day. Doses may be administered until the desired therapeutic effect is achieved or indefinitely to maintain the desired therapeutic effect. Doses of compounds of the invention may be in the range of 0.001 mg/kg to 100 mg/kg, such as 0.001 mg/kg to 50 mg/kg. Preferably, the selected dose is administered orally or intravenously.

Methods, Uses, Compounds For Use in Manufacture and/or Treatment of Diseases

In general, the invention also relates to uses of the compounds and/or pharmaceutical compositions of the invention as defined herein for use as a medicament or for use in therapy.

Compounds of the invention as defined herein are inhibitors of voltage-gated sodium ion channels, and particularly the voltage-gated sodium ion channel Nav1.8. The activity of a compound utilized in this invention as an inhibitor of Nav1.8 may be assayed according to methods described generally in the Examples herein, or according to methods available to one of ordinary skill in the art.

Accordingly, in one aspect, the invention relates to uses of compounds and pharmaceutical compositions of the invention as inhibitors of voltage-gated sodium ion channels, particularly Nav1.8.

In one embodiment, the invention relates to a method of inhibiting a voltage-gated sodium ion channel in a subject in need thereof, comprising administering to the subject an effective amount of a compound of the invention or a pharmaceutical composition of the invention as described herein. In one embodiment, the voltage-gated sodium channel is Nav1.8.

In embodiment, the invention relates to a compound of the invention or a pharmaceutical composition of the invention for use in inhibiting a voltage-gated sodium ion channel. In one embodiment, the voltage-gated sodium channel is Nav1.8.

In one embodiment, the invention relates to use of a compound of the invention or a pharmaceutical composition of the invention in the manufacture of a medicament for inhibiting a voltage-gated sodium ion channel. In one embodiment, the voltage-gated sodium channel is Nav1.8.

Without wishing to be bound by any particular theory, the compounds and compositions of the invention are particularly useful for treating a disease, condition, or disorder where activation or hyperactivity of Nav1.8 is implicated in the disease, condition, or disorder. When activation or hyperactivity of Nav1.8 is implicated in a particular disease, condition, or disorder, the disease, condition, or disorder may also be referred to as a “Nav1.8-mediated disease, condition or disorder.” Exemplary Nav1.8-mediated diseases, disorders, and conditions include pain and pain-associated diseases, disorders, and conditions, and cardiovascular diseases, disorders, and conditions such as atrial fibrillation.

Thus, in another aspect, the invention relates to uses of compounds and pharmaceutical compositions of the invention in methods and medicaments for treating pain or a pain-associated disease, disorder, or condition and/or for treating cardiovascular diseases, disorders, and conditions.

As used herein, “patient” or “subject” in need thereof refers to a human or mammal. The term “mammal” as used herein, encompasses any mammal. Examples of mammals include, but are not limited to, cows, horses, sheep, pigs, cats, dogs, mice, rats, rabbits, guinea pigs, and non-human primates (NHPs), such as monkeys or apes, humans, etc. Suitably the subject being treated is a human.

As used herein, the terms “treat”, “treating”, and/or “treatment” used in reference to a disease, disorder, or condition mean to ameliorate or prevent the condition or one or more biological manifestations of the condition; to interfere with one or more points in the biological cascade that leads to or is responsible for the condition; to alleviate one or more of the symptoms or effects associated with the condition; to slow the progression of the condition or one or more of the biological manifestations of the condition; or to lessen the severity of the condition or one or more symptoms or effects associated with the condition. As mentioned above, “treatment” of a disease, disorder, or condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “effective amount” and “therapeutically effective amount” are used interchangeably. An effective amount in reference to a compound of the invention means an amount of the compound sufficient to treat the patient's condition, but low enough to avoid serious side effects (at a reasonable benefit/risk ratio) within the scope of sound medical judgment. An effective amount of a compound or pharmaceutically acceptable salt thereof and/or corresponding tautomer form thereof of the invention or corresponding pharmaceutical composition thereof will vary according to factors, such as the particular compound chosen (e.g., consider the potency, efficacy, and half-life of the compound); the route of administration chosen; the condition being treated; the severity of the condition being treated; the age, size, weight, and physical condition of the patient or subject being treated; the medical history of the patient or subject being treated; the duration of the treatment; the nature of concurrent therapy; the desired therapeutic effect, etc.

According to embodiments of the invention, a pain-associated disease, disorder or condition is pain caused by any one of a variety of diseases of varying etiologies as described throughout the present disclosure. In some embodiments, pain or a pain-associated disease, disorder, or condition is neuropathic pain, chronic pain, acute pain, nociceptive pain, inflammatory pain, musculoskeletal pain, visceral pain, cancer pain, idiopathic pain, multiple sclerosis, Charcot-Marie-Tooth syndrome, or incontinence.

In some embodiments, pain or a pain-associated disease, disorder, or condition is acute pain.

In some embodiments, pain or a pain-associated disease, disorder, or condition is neuropathic pain or chronic neuropathic pain.

In some embodiments, pain or a pain-associated disease, disorder, or condition is neuropathic pain or chronic neuropathic pain selected from small fiber neuropathy, small fiber-mediated diabetic neuropathy, idiopathic small fiber neuropathy, painful diabetic neuropathy or polyneuropathy.

In some embodiments pain or a pain-associated disease, disorder, or condition is neuropathic pain selected from post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion, complex regional pain syndrome, drug therapy induced neuralgia, cancer chemotherapy induced neuralgia, anti-retroviral therapy induced neuralgia, post spinal cord injury pain, idiopathic small-fiber neuropathy, idiopathic sensory neuropathy or trigeminal autonomic cephalalgia.

In some embodiments, pain or a pain-associated disease, disorder, or condition is neuropathic pain or chronic neuropathic pain selected from diabetic peripheral neuropathy, pain caused by neuropathy, neurologic or neuronal injury, pain associated nerve injury, neuralgias and associated acute or chronic pain, post-herpetic neuralgia, pain associated root avulsions, painful traumatic mononeuropathy, painful polyneuropathy, erythromelalgia, paroxysmal extreme pain disorder (PEPD), burning mouth syndrome, central pain syndromes caused by a lesion at a level of nervous system, traumatic nerve injury, nerve compression or entrapment, congenital insensitivity to pain (CIP), dysmenorrheal, primary erythromelalgia, HIV peripheral sensory neuropathy, pudendal neuralgia, spinal nerve injury, chronic inflammatory demyelinating polyneuropathy (CIDP), carpal tunnel syndrome and vasculitic neuropathy.

In some embodiments, pain or a pain-associated disease, disorder, or condition is visceral pain, wherein visceral pain is inflammatory bowel disease pain, Crohn's disease pain or interstitial cystitis pain.

In some embodiments, pain or a pain-associated disease, disorder, or condition is musculoskeletal pain, wherein musculoskeletal pain is osteoarthritis pain, back pain, cold pain, burn pain or dental pain.

In some embodiments, pain or a pain-associated disease, disorder, or condition is idiopathic pain, wherein idiopathic pain is fibromyalgia pain.

In some embodiments, pain or a pain-associated disease, disorder, or condition is chronic or acute pre-operative associated pain or chronic or acute post-operative associated pain. Post-operative associated pain includes ambulatory post-operative pain. Ambulatory surgery, also known as outpatient surgery, refers to same day surgery that does not require an overnight stay in a hospital or other medical facility. In some embodiments, pre-operative associated pain is selected from neuropathic pain or chronic neuropathic pain, chronic osteoarthritis pain, dental pain or inflammatory pain. In some embodiments, post-operative associated pain is selected from bunionectomy pain, hernia repair pair, breast surgery pain or cosmetic surgical pain.

In some embodiments, pain or a pain-associated disease, disorder, or condition is pain caused by trauma or iatrogenic medical or dental procedures. As used herein, the term “iatrogenic” refers to pain induced inadvertently by a medical or dental personnel, such as surgeon or dentist, during medical or dental treatment(s) or diagnostic procedure(s), which include, but are not limited to pain caused by pre-operative (i.e., “before”), peri-operative (i.e., “during” or medically induced pain during non-surgical or operative treatment(s)) and post-operative (i.e., after, post-operative or surgical induced caused pain) medical or dental procedures.

In some embodiments, pain or a pain-associated disease, disorder, or condition is nociceptive pain, wherein nociceptive pain is post-surgical pain, cancer pain, back and craniofacial pain, osteoarthritis pain, dental pain or diabetic peripheral neuropathy.

In some embodiments, pain or a pain-associated disease, disorder, or condition is inflammatory pain. Inflammatory pain can be pain of varied physiological origins. In some embodiments, inflammatory pain is selected from pain associated with osteoarthritis, rheumatoid arthritis, rheumatic disorder, teno-synovitis and gout, shoulder tendonitis or bursitis, gouty arthritis, and polymyalgia rheumatica, primary hyperalgesia, secondary hyperalgesia, primary allodynia, secondary allodynia, or other pain caused by central sensitization; complex regional pain syndrome, chronic arthritic pain and related neuralgias or acute pain. In some embodiments inflammatory pain is selected from pain associated with rheumatoid arthritis, osteoarthritis, rheumatoid spondylitis, gouty arthritis orjuvenile arthritis. In some embodiments, inflammatory pain is selected from rheumatoid arthritis; rheumatoid spondylitis; gouty arthritis; juvenile arthritis; rheumatic disorder; gout; shoulder tendonitis or bursitis; polymyalgia rheumatica; primary hyperalgesia; secondary hyperalgesia; primary allodynia; secondary allodynia; or other pain caused by central sensitization, complex regional pain syndrome, chronic or acute arthritic pain and related neuralgias.

In some embodiments, inflammatory pain is selected from rheumatoid arthritis pain or vulvodynia.

In some embodiments, the inflammatory pain is selected from osteoarthritis, chronic osteoarthritis pain (e.g., hip or knee) or chronic inflammatory demyelinating polyneuropathy.

In some embodiments pain or a pain-associated disease, disorder, or condition is musculoskeletal pain. In some embodiments, musculoskeletal pain is selected from bone and joint pain, osteoarthritis; lower back and neck pain; pain resulting from physical trauma or amputation. In some embodiments, musculoskeletal pain is selected from bone and joint pain, osteoarthritis (e.g., knee, hip), tendonitis (e.g., shoulder), bursitis (e.g., shoulder) tenosynovitis, lower back and neck pain, sprains, strains, or pain resulting from physical trauma or amputation.

In some embodiments, pain or a pain-associated disease, disorder, or condition is neurologic or neuronal injury associated or related pain disorders caused by diseases selected from neuropathy, pain associated nerve injury, pain associated root avulsions, painful traumatic mononeuropathy, painful polyneuropathy, erythromelalgia, paroxysmal extreme pain disorder (PEPD), burning mouth syndrome; central pain syndromes caused by a lesion at a level of nervous system); traumatic nerve injury, nerve compression or entrapment, congenital insensitivity to pain (CIP), dysmenorrheal, primary erythromelalgia; HIV peripheral sensory neuropathy; pudendal neuralgia, spinal nerve injury, chronic inflammatory demyelinating polyneuropathy (CIDP), carpal tunnel syndrome or vasculitic neuropathy.

In some embodiments, pain or a pain-associated disease, disorder, or condition is pain caused by trauma, or pain caused by iatrogenic, medical, or dental procedures.

In some embodiments, pain or a pain-associated disease, disorder, or condition is myofascial pain; myositis or muscle inflammation; repetitive motion pain; complex regional pain syndrome; sympathetically maintained pain; cancer, toxins and chemotherapy related pain; postsurgical pain syndromes and/or associated phantom limb pain; post-operative medical or dental procedures or treatments pain; pain associated with HIV or pain induced by HIV treatment.

In some embodiments, pain or a pain-associated disease, disorder, or condition is neuropathic pain or other pain-associated disease, disorder, or condition selected from peripheral neuropathic pain, central neuropathic pain, inherited erythromelalgia (IEM), small fiber neuralgia (SFN), paroxysmal extreme pain disorder (PEPD), painful diabetic neuropathy, chronic lower back pain, neuropathic back pain, sciatica, non-specific lower back pain, multiple sclerosis pain, HIV-related neuropathy, post-herpetic neuralgia, trigeminal neuralgia, vulvodynia, pain resulting from physical trauma, post-limb amputation pain, neuroma pain, phantom limb pain, cancer, toxins, or chronic inflammatory conditions.

In some embodiments, pain or a pain-associated disease, disorder, or condition is acute pain, chronic pain, neuropathic pain, inflammatory pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, dipolar disorder, myotonia, arrhythmia, movement disorders, neuroendocrine disorders, ataxia, multiple sclerosis, irritable bowel syndrome, incontinence, visceral pain, osteoarthritis pain, postherpetic neuralgia, diabetic neuropathy, radicular pain, sciatica, back pain, head pain, neck pain, severe pain, intractable pain, nociceptive pain, breakthrough pain, postsurgical pain, cancer pain, stroke, cerebral ischemia, traumatic brain injury, amyotrophic lateral sclerosis, stress induced angina, exercise induced angina, palpitations, hypertension, or abnormal gastro-intestinal motility.

In some embodiments, pain or a pain-associated disease, disorder, or condition is femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache, including, cluster headaches; chronic and acute neuropathic pain, post-herpetic neuralgia; diabetic neuropathy; HIV-associated neuropathy; trigeminal neuralgia; Charcot-Marie Tooth neuropathy; hereditary sensory neuropathies; peripheral nerve injury; painful neuromas; ectopic proximal and distal discharges; radiculopathy; chemotherapy induced neuropathic pain; radiotherapy-induced neuropathic pain; post-mastectomy pain; central pain; spinal cord injury pain; post-stroke pain; thalamic pain; complex regional pain syndrome; phantom pain; intractable pain; acute pain, acute post-operative pain; acute musculoskeletal pain; joint pain; mechanical low back pain; neck pain; tendonitis; injury/exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory, burn and trauma pain; acute intermittent pain, endometriosis; acute herpes zoster pain; sickle cell anemia; acute pancreatitis; breakthrough pain; orofacial pain including sinusitis pain, dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease, including, urinary incontinence; hyperactivity bladder; painful bladder syndrome; interstitial cyctitis (IC); prostatitis; complex regional pain syndrome (CRPS), type I and type II; widespread pain, paroxysmal extreme pain, pruritis, tinnitis, or angina-induced pain.

In another aspect, the invention relates to uses of compounds and pharmaceutical compositions of the invention in methods and medicaments for treating cardiovascular diseases, disorders and conditions, including atrial fibrillation and cardiac arrhythmias.

In some embodiments, the cardiovascular disease is atrial fibrillation that is either idiopathic in nature or caused by a disease as defined herein. Atrial fibrillation can be paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease. In particular embodiments, atrial fibrillation is selected from paroxysmal, sustained, or long-standing atrial fibrillation.

In some embodiments, the cardiovascular disease includes cardiac arrhythmias.

In one aspect, the invention relates to a method of treatment of pain or a pain-associated disease, disorder, or condition as defined herein in a subject in need thereof, comprising administering to the subject a therapeutically effective amount of a compound of the invention or a pharmaceutical composition of the invention as described herein.

In an embodiment, provided is a method of treatment of acute pain or chronic pain in a subject in need thereof, comprising administering to the subject a therapeutically effective amount of a compound of the invention or a pharmaceutical composition of the invention as described herein.

In an embodiment, provided is a method of treatment of acute pain in a subject in need thereof, comprising administering to the subject a therapeutically effective amount of a compound of the invention or a pharmaceutical composition of the invention as described herein.

In an embodiment, provided is a method of treatment of pain caused by trauma; pain caused by iatrogenic medical or dental procedures; or pre-operative or post-operative associated pain in a subject in need thereof, comprising administering to the subject a therapeutically effective amount of a compound of the invention or a pharmaceutical composition of the invention as described herein.

In an embodiment, provided is a method of treatment of neuropathic pain, nociceptive pain, inflammatory pain, musculoskeletal pain, visceral pain, or idiopathic pain in a subject in need thereof, comprising administering to the subject a therapeutically effective amount of a compound of the invention or a pharmaceutical composition of the invention as described herein.

In an embodiment, provided is a method of treatment of neuropathic pain or chronic neuropathic pain selected from small fiber neuropathy, small fiber-mediated diabetic neuropathy, idiopathic small fiber neuropathy, painful diabetic neuropathy or polyneuropathy in a subject in need thereof, comprising administering to the subject a therapeutically effective amount of a compound of the invention or a pharmaceutical composition of the invention as described herein.

In an embodiment, provided is a method of treatment of inflammatory pain selected from osteoarthritis, chronic osteoarthritis pain, or chronic inflammatory demyelinating polyneuropathy in a subject in need thereof, comprising administering to the subject a therapeutically effective amount of a compound of the invention or a pharmaceutical composition of the invention as described herein.

In one aspect, the invention relates to a method of treatment of atrial fibrillation as defined herein in a subject in need thereof, comprising administering to the subject a therapeutically effective amount of a compound of the invention or a pharmaceutical composition of the invention as described herein.

In one embodiment, provided is a method of treatment of atrial fibrillation, wherein the atrial fibrillation is paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease.

In another aspect, the invention provides compounds of the invention and pharmaceutical compositions of the invention as described herein for use in treatment of pain or a pain-associated disease, disorder, or condition as defined herein.

In an embodiment, provided is a compound of the invention or pharmaceutical composition of the invention for use in treatment of acute pain or chronic pain.

In an embodiment, provided is a compound of the invention or pharmaceutical composition of the invention for use in treatment of acute pain.

In an embodiment, provided is a compound of the invention or pharmaceutical composition of the invention for use in treatment of pain caused by trauma; pain caused by iatrogenic medical or dental procedures; or pre-operative or post-operative associated pain.

In an embodiment, provided is a compound of the invention or pharmaceutical composition of the invention for use in treatment of neuropathic pain, nociceptive pain, inflammatory pain, musculoskeletal pain, visceral pain, or idiopathic pain.

In an embodiment, provided is a compound of the invention or pharmaceutical composition of the invention for use in treatment of neuropathic pain or chronic neuropathic pain selected from small fiber neuropathy, small fiber-mediated diabetic neuropathy, idiopathic small fiber neuropathy, painful diabetic neuropathy or polyneuropathy.

In an embodiment, provided is a compound of the invention or pharmaceutical composition of the invention for use in treatment of inflammatory pain selected from osteoarthritis, chronic osteoarthritis pain, or chronic inflammatory demyelinating polyneuropathy.

In another aspect, the invention relates to a compound of the invention or a pharmaceutical composition of the invention for use in treatment of atrial fibrillation.

In one embodiment, provided is a compound of the invention or pharmaceutical composition of the invention for use in treatment of atrial fibrillation, wherein the atrial fibrillation is paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease.

In another aspect, the invention also provides uses of compounds of the invention or pharmaceutical compositions of the invention as described herein in the manufacture of a medicament for treatment of pain and pain associated diseases, disorders, and conditions as described herein.

In an embodiment, provided is use of a compound of the invention or pharmaceutical composition of the invention in the manufacture of a medicament for treatment of acute pain or chronic pain.

In an embodiment, provided is use of a compound of the invention or pharmaceutical composition of the invention in the manufacture of a medicament for treatment of acute pain.

In an embodiment, provided is use of a compound of the invention or pharmaceutical composition of the invention in the manufacture of a medicament for treatment of pain caused by trauma; pain caused by iatrogenic medical or dental procedures; or pre-operative or post-operative associated pain.

In an embodiment, provided is use of a compound of the invention or pharmaceutical composition of the invention in the manufacture of a medicament for treatment of neuropathic pain, nociceptive pain, inflammatory pain, musculoskeletal pain, visceral pain, or idiopathic pain.

In an embodiment, provided is use of a compound of the invention or pharmaceutical composition of the invention in the manufacture of a medicament for treatment of neuropathic pain or chronic neuropathic pain selected from small fiber neuropathy, small fiber-mediated diabetic neuropathy, idiopathic small fiber neuropathy, painful diabetic neuropathy or polyneuropathy.

In an embodiment, provided is use of a compound of the invention or pharmaceutical composition of the invention in the manufacture of a medicament for treatment of inflammatory pain selected from osteoarthritis, chronic osteoarthritis pain, or chronic inflammatory demyelinating polyneuropathy.

In another aspect, the invention also provides uses of compounds of the invention or pharmaceutical compositions of the invention as described herein in the manufacture of a medicament for treatment of atrial fibrillation.

In an embodiment, provided is use of a compound of the invention or pharmaceutical composition of the invention in the manufacture of a medicament for treatment of atrial fibrillation, wherein the atrial fibrillation is paroxysmal atrial fibrillation, sustained atrial fibrillation, long-standing atrial fibrillation, atrial fibrillation with heart failure, atrial fibrillation with cardiac valve disease, or atrial fibrillation with chronic kidney disease.

In another aspect, the invention relates to a compound of the invention or a pharmaceutical composition of the invention as described herein for use in therapy.

### Combination Therapies and Uses for Therapy

Compounds and pharmaceutical compositions of the invention as described herein can be used in combination with one or more additional therapeutic agents. Such additional therapeutic agents can be administered concurrently with, prior to, or subsequent to treatment with a compound or pharmaceutical composition of the invention as described herein.

In the context of this specification, the term “concurrently” when referring to simultaneous administration of compounds or therapeutic agents means at the same time, as would be the case, for example in embodiments where a compound and additional therapeutic agent(s) are combined in a single preparation, or when a compound and additional therapeutic agent(s) are administered separately but taken within a short duration or period of time.

In light of the foregoing, the invention also relates to a combination therapy, which may be a comprised of a simultaneous or co-administration, or serial administration of a combination of compounds or pharmaceutical compositions of the invention with one or more additional therapeutic agents. Such combination therapy can be used for treatment of pain or any pain-associated disease, disorder, or condition, or a cardiovascular disease, disorder, or condition as defined throughout the present specification.

Therapeutic agents suitable for use in combination with the compounds and pharmaceutical compositions of the invention include, but are not limited to: Acetaminophen, Acetylsalicylic acid, Nav1.7 Inhibitors, Nav1.9 Inhibitors, anti-depressants (i.e. such as, but not limited to duloxetine or amitriptyline), anti-convulsants (i.e. such as, but not limited to pregabalin and gabapentin), opiates (i.e., such as, but not limited to hydrocodone, codeine, morphine, oxycodone, oxymorphone, fentanyl, and the like), etc.; and where administration of the above, respectively, also is determined by one of ordinary skill in the art. In one aspect, suitable Nav1.7 Inhibitors or Nav1.9 Inhibitors for use in the invention, include, but are not limited to those Nav1.7 Inhibitors or Nav1.9 Inhibitors known in the chemical literature.

Each component of a combination used for therapeutic purposes (e.g., compound or pharmaceutical composition of the invention and additional therapeutic agent) may be administered orally, intravenously or parenterally or in combinations thereof. Each component of a therapeutic combination may be, but is not limited to being administered by simultaneous administration, co-administration, or serial administration; and/or by identical or different routes of administration or combinations of administration routes. In certain embodiments, each identical or different route of administration or combinations of administration routes is selected from oral, intravenous or parenteral administration.

### EXAMPLES

The following examples illustrate the invention. These examples are not intended to limit the scope of the present invention, but rather to provide guidance to the skilled artisan to prepare and use the compounds, compositions, and methods of the present invention.

While particular aspects or embodiments of the present invention are described, the skilled artisan will appreciate that various changes and modifications can be made without departing from the spirit and scope of the invention.

### Synthesis Examples

It will be understood by the skilled artisan that purification methods (using acidic or basic modifiers) or compound workup procedures (using acidic or basic conditions) may result in formation of a salt of a title compound (for example, hydrobromic acid, formic acid, hydrochloric acid, trifluoroacetic acid, or ammonia salts of a title compound). The invention is intended to encompass such salts.

Final compounds were characterized with GCMS and LCMS (conditions listed below) and NMR. ¹H NMR or ¹⁹FNMR spectra were recorded using a BrukerAvance III 500 MHz spectrometer, Bruker Avance 400 MHz spectrometer and Varian Mercury Plus-300 MHz spectrometer. CDCl₃ is deuteriochloroform, DMSO-d₆ is hexadeuteriodimethylsulfoxide, and CD₃OD is tetradeuteriomethanol. Chemical shifts are reported in parts per million (ppm) downfield from the internal standard tetramethylsilane (TMS) or the NMR solvent.

Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

### Analytical Methods:

1) LCMS Method: Acquity UPLC with Waters Acquity QDa mass detector using electrospray positive [ES+ve to give M+H⁺] equipped with a CSH C18 column (30 mm×2.1 mm, i.d. 1.7 μm packing diameter) at 45° C. eluting with 0.1% TFA in water (solvent A) and 0.1% TFA in acetonitrile (solvent B), using the following elution gradient: 1-100% (solvent B) over 1.85 min at a flow rate of 1.3 ml/min.

2) LCMS Method: Acquity UPLC with Waters Acquity QDa mass detector using electrospray positive [ES+ve to give M+H⁺] equipped with a CSH C18 column (30 mm×2.1 mm, i.d. 1.7 μm packing diameter) at 45° C. eluting with formic acid in Water (solvent A) and formic acid in acetonitrile (solvent B), using the following elution gradient: 1-100% (solvent B) over 1.85 min at a flow rate of 1.3 ml/min.

3) LCMS Method: Acquity UPLC with Waters Acquity QDa mass detector using electrospray positive [ES+ve to give M+H⁺] equipped with a CSH C18 column (30 mm×2.1 mm, i.d. 1.7 μm packing diameter) at 45° C. eluting with 10 mM ammonium bicarbonate in water adjusted to pH=10 with 25% ammonium hydroxide solution (solvent A) and acetonitrile (solvent B), using the following elution gradient: 1-100% (solvent B) over 1.85 min at a flow rate of 1.3 ml/min.

4) LCMS method: Agilent 1290 Infinity II LC system with Agilent MSD 6125B/6130 using multi mode (ESI and APCI+ve and −ve) equipped with a Sunfire C18 column (30 mm×2.1 mm, i.d. 3.5 μm packing diameter) at 25° C. eluting with 0.1% formic acid in water (solvent A) and 0.1% formic acid in acetonitrile (solvent B), using the following elution gradient: 0-100% (solvent B) over 3.1 min and holding at 100% for 0.8 min at a flow rate of 1.0 ml/min.

5) LCMS method: Agilent 1290 Infinity II LC system with Agilent MSD 6125B/6130 using multi mode (ESI and APCI+ve and −ve) equipped with a Atlantis dC18 column (50 mm×4.6 mm, i.d. 5 μm packing diameter) at 25° C. eluting with 0.1% TFA in water (solvent A) and methanol (solvent B), using the following elution gradient: 5-95% (solvent B) over 5.0 min and holding at 95% for 1.5 min at a flow rate of 1.0 ml/min.

6) LCMS method: Agilent 1290 Infinity II LC system with Agilent MSD 6125B/6130 using multi mode (ESI and APCI+ve and −ve) equipped with a Zorbax XDB C18 column (50 mm×4.6 mm, i.d. 3.5 μm packing diameter) at 25° C. eluting with 10 mM ammonium acetate in water (solvent A) and acetonitrile (solvent B), using the following elution gradient: Solvent B: 10-95% (solvent B) over 3.5 min and holding at 95% for 1.0 min at a flow rate of 1.0 ml/min.

7) LCMS method: Agilent 1290 Infinity II LC system with Agilent MSD 6125B/6130 using multi mode (ESI and APCI+ve and −ve) equipped with a Xbridge C8 column (50 mm×4.6 mm, i.d. 3.5 μm packing diameter) at 25° C. eluting with 10 mM ammonium bicarbonate in water (solvent A) and acetonitrile (solvent B), using the following elution gradient: 10-95% (solvent B) over 4.0 min and holding at 95% for 1.0 min at a flow rate of 1.0 ml/min. 8) GCMS Method: Agilent 7890B GC system with Agilent MSD 5977B using EI equipped with a HP-5 column (30 m×0.32 mm, 0.25 μm film thickness) at 250° C. eluting with helium at a flow rate of 2 mL/min and 10 min run time under the following chromatographic run conditions: 120° C. for 1 min, 40° C./min up to 300° C., hold for 4.5 min.

In the following experimental descriptions, the following abbreviations may be used:

| Abbreviation | Meaning |
|---|---|
| AcOH | acetic acid |
| aq. | aqueous |
| BBr₃ | boron tribromide |
| BCl₃ | boron trichloride |
| BH₃ | borane |
| BINAP | 2,2′-bis(diphenylphosphino)-1,1′-binaphthalene |
| Bn | benzyl |
| brine | saturated aqueous sodium chloride |
| BuLi or nBuLi | butyllithium |
| CDI | carbonyldiimidazole |
| CH₂Cl₂ | methylene chloride |
| CH₃CN | acetonitrile |
| COCl₂ | oxalyl chloride |
| CS₂CO₃ | cesium carbonate |
| DABCO | 1,4-diazabicyclo[2.2.2]octane |
| DCC | dicyclohexylcarbodiimide |
| DCM or CH₂Cl₂ | methylene chloride |
| DEAD | diethyl azodicarboxylate |
| DEAP | diethyl aminopyridine |
| DIAD | diisopropyl azodicarboxylate |
| DIPEA, DIEA, | N,N-diisopropylethylamine |
| Hunig's base | |
| DMA | Dimethylacetamide |
| DMAP | 4-dimethylaminopyridine |
| DMF | N,N-dimethylformamide |
| DME | dimethoxyethane |
| DMSO | dimethylsulfoxide |
| EDC | 1-[3-(dimethylamino)propyl]-3-ethylcarbodiimide |
| | hydrochloride |
| Et | ethyl |
| Et₃N | triethylamine |
| Et₂O | diethyl ether |
| EtOAc | ethyl acetate |
| EtOH | ethanol |
| Fmoc or fmoc | fluorenylmethyloxycarbonyl |
| g, G, gm, GM | gram |
| GCMS | gas chromatography-mass spectroscopy |
| h or hr | hour(s) |
| H₂ | hydrogen |
| H₂O₂ | hydrogen peroxide |
| H₂O | water |
| H₂SO₄ | sulfuric acid |
| HATU | (O-(7-azabenzotriazol-1-yl)-N,N,N′,N′- |
| | tetramethyluronium hexafluorophosphate) |
| HBTU | 2-(1H-benzo[d][1,2,3]triazol-1-yl)-1,1,3,3- |
| | tetramethylisouronium hexafluorophosphate(V) |
| HCl | hydrochloric acid |
| HCO₂H | formic acid |
| HOBt or HOBT | 1-hydroxybenzotriazole |
| HPLC | high performance liquid chromatography |
| I₂ | Iodine |
| JLR | jacketed lab reactor |
| K₂CO₃ | potassium carbonate |
| KHSO₄ | potassium hydrogen sulfate |
| KOAc | potassium acetate |
| L or l | liter |
| LAH | lithium aluminum hydride |
| LCMS | liquid chromatography-mass spectroscopy |
| LDA | lithium diisopropyl amide |
| LED | light-emitting diode |
| LiOH | lithium hydroxide |
| LHMDS | lithium bis(trimethylsilyl)amide |
| mCPBA | meta-chloroperoxybenzoic acid |
| MDAP | mass directed autopurification |
| Me | methyl |
| MeOH | methanol |
| mg, MG | milligram |
| MgBr₂ | magnesium bromide |
| MgSO₄ | magnesium sulfate |
| Min or mins | minute(s) |
| ml or mL or ML | milliliter |
| mmol | millimole |
| MnO₂ | manganese dioxide |
| Mol, mol | mole |
| MS | mass spectrum |
| MTBE | methyl tert-butyl ether |
| μw | microwave |
| N₂ | nitrogen |
| Na(CN)BH₃ | sodium cyanoborohydride |
| NaCl | sodium chloride |
| Na₂CO₃ | sodium carbonate |
| NaHCO₃ | sodium bicarbonate |
| NaHMDS | sodium bis(trimethylsilyl)amide |
| NaHSO₃ | sodium bisulfite |
| NaH | sodium hydride |
| NaI | sodium iodide |
| NaOH | sodium hydroxide |
| Na₂SO₃ | sodium sulfite |
| Na₂SO₄ | sodium sulfate |
| NH₄Cl | ammonium chloride |
| HCO₂•NH₄ | ammonium formate |
| NH₄OH | ammonium hydroxide |
| nm | nanometer |
| NMO | 4-methylmorpholine N-oxide |
| NMP | N-methyl-2-pyrrolidone |
| Pd/C | palladium on carbon |
| PdCl₂(dbpf) | 1,1′-bis(di-tert-butylphosphino)ferrocene |
| | dichloropalladium |
| Pd(dppf)Cl₂/ | [1,1′-bis(diphenylphosphino)ferrocene] |
| PdCl₂(dppf) | dichloropalladium(II) |
| PdCl₂(dppf)- | [1,1′-bis(diphenylphosphino)ferrocene] |
| CH₂Cl₂ adduct | dichloropalladium(II), complex with dichloromethane |
| Pd₂ (dba)₃ | tris(dibenzylideneacetone)dipalladium(0) |
| Pd(Ph₃)₄, | tetrakis(triphenylphosphine)palladium(0) |
| tetrakis | |
| PdOAc₂ or | palladium acetate |
| Pd(OAc)₂ | |
| Pd(OH)₂ | palladium hydroxide |
| PIFA | [Bis(trifluoroacetoxy)iodo]benzene |
| Ph | phenyl |
| PL HCO₃ MP | macroporus polystyrene supported carbonate |
| POCl₃ | phosphoryl chloride |
| psi | Pounds per square inch |
| PTFE | polytetrafluoroethylene |
| PTSOH or | p-Toluenesulfonic acid |
| PTSA or | |
| pTsOH | |
| rt or RT | room temperature |
| sat. | saturated |
| SFC | supercritical fluid chromatography |
| Si | silica |
| Si SPE | silica gel cartridges |
| SiO₂ | silica gel |
| SPE | solid phase extraction |
| T3P ® | propylphosphonic anhydride |
| tBu or t-Bu | tert-butyl group |
| TBAB | tetrabutylammonium bromide |
| TBAF | tetrabutylammonium fluoride |
| TBAI | tetrabutylammonium iodide |
| TBDMSCI | tert-butyldimethylsilyl chloride |
| TBME | tert-butylmethyl ether |
| TBTU | 2-(1H-benzotriazole-1-yl)-1,1,3,3-tetramethyluronium |
| | tetrafluoroborate |
| TEA | triethylamine |
| TFA | trifluoroacetic acid |
| THF | tetrahydrofuran |
| TiCl₄ | titanium tetrachloride |
| TMS-Br or | trimethylsilyl bromide |
| TMSBr | |
| TMS-Cl or | trimethylsilyl chloride |
| TMSCl | |
| TMSI | lodotrimethylsilane or trimethylsilyl iodide |
| TMS-OTf | trimethylsilyl triflate |
| or TMSOtf | |
| tR | retention time |
| UPLC | ultra performance liquid chromatography |
| Xantphos | 4,5-Bis(diphenylphosphino)-9,9-dimethylxanthene |
| Xphos | 2-Dicyclohexylphosphino-2′,4′,6′-triisopropylbiphenyl |

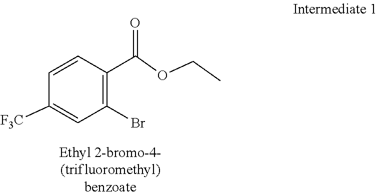 [SMILES: CCOC(=O)c1ccc(C(F)(F)F)cc1Br]

To a stirring solution of 2-bromo-4-(trifluoromethyl)benzoic acid (10.0 g, 37.2 mmol) in DMF (100 ml-) was added K₂CO₃ (5.65 g, 40.9 mmol) followed by ethyl iodide (3.60 mL, 44.6 mmol) dropwise under N₂ at 25° C. The reaction mixture was stirred at the same temperature for 3 hours. Water (150 ml-) was added and the reaction was extracted with EtOAc (2×250 mL). The combined organic extracts were washed with brine (150 mL), dried over Na₂SO₄, filtered, and concentrated. The residue was purified by flash column chromatography (Biotage, 100 g SNAP column, 10% EtOAc/petroleum ether over 40 minutes) to give the title compound as a colorless oil (9.3 g, 31.3 mmol, 84% yield). GCMS (m/z) 296.0 (M)⁺.

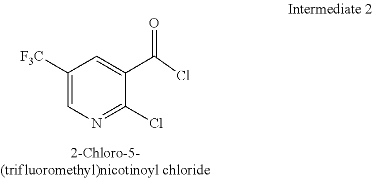 [SMILES: O=C(Cl)c1cc(C(F)(F)F)cnc1Cl]

To a stirred solution of 2-chloro-5-(trifluoromethyl)nicotinic acid (50 g, 222 mmol) in DCM (500 mL) were added oxalyl chloride (23.28 mL, 266 mmol) and DMF (1.716 mL, 22.17 mmol) at 0° C. and the reaction mixture was stirred for 1 hour at RT. The reaction mixture was concentrated under reduced pressure to dryness under N₂ to yield the title compound as a brown gum (52 g, 213 mmol, 96% yield).

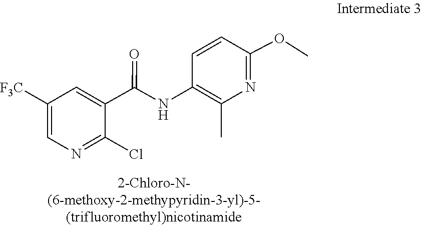 [SMILES: COc1ccc(NC(=O)c2cc(C(F)(F)F)cnc2Cl)c(C)n1]

A mixture of 6-methoxy-2-methylpyridin-3-amine (32.4 g, 234 mmol) and TEA (89 mL, 639 mmol) in DCM (300 mL) was added to a stirred solution of 2-chloro-5-(trifluoromethyl)nicotinoyl chloride (52 g, 213 mmol) in DCM (300 mL) at 0° C. and the reaction mixture was stirred for 2 hours. The reaction mixture was quenched with ice-cold water (500 mL) and extracted with DCM (3×500 mL). The combined organic phases were washed with water (500 mL) and brine (500 mL), dried over Na₂SO₄ and concentrated in vacuo. The crude product was purified by column chromatography (Biotage, 340 g SiO₂ column, 0-30% EtOAc/petroleum ether over 40 minutes) to give the title compound as a brown solid (37 g, 106 mmol, 49.9% yield). MS (m/z) 346.0 (M+H⁺).

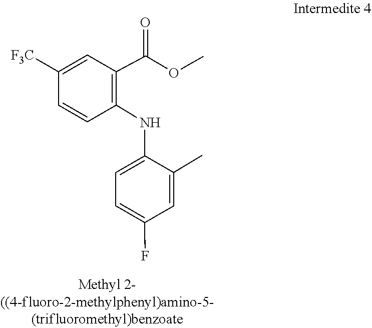 [SMILES: COC(=O)c1cc(C(F)(F)F)ccc1Nc1ccc(F)cc1C]

To a solution of methyl 2-bromo-5-(trifluoromethyl)benzoate (2 g, 7.1 mmol) and 4-fluoro-2-methylaniline (1.06 g, 8.48 mmol) in 1,4-dioxane (20 mL) under nitrogen at room temperature was added cesium carbonate (4.60 g, 14.13 mmol) and BINAP (0.44 g, 0.71 mmol) in a one charge. The reaction mixture was purged with nitrogen for 10 min, then Pd₂(dba)₃ (0.324 g, 0.353 mmol) was added into the reaction mixture. The reaction mixture was stirred at 100° C. for 16 h. The reaction mixture was cooled to RT and filtered through a Celite @pad and the filtrate was concentrated onto SiO₂. Purification by flash chromatography on SiO₂ (25 g) with 0-30% EtOAc in petroleum ether as eluant afforded the title compound as a colorless solid (2.3 g, 7.0 mmol, 99% yield). MS (m/z) 328.0 (M+H⁺).

Intermediates 5-6 were prepared from the indicated aryl halogen and aniline by methods analogous to those described for Intermediate 4.

| | | | | Aryl | |
|---|---|---|---|---|---|
| Int. | Name | Structure | Characterization | halogen | Aniline |
| 5 | ethyl 2-((4- fluoro-2- methylphenyl) amino)-4- (trifluoromethyl) benzoate | 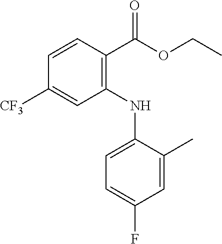 [SMILES: CCOC(=O)c1ccc(C(F)(F)F)cc1Nc1ccc(F)cc1C] | MS (m/z) 342.0 (M + H⁺) | ethyl 2- bromo-4- (trifluoro- methyl) benzoate | 4-fluoro-2- methyl- aniline |
| 6 | methyl 5-chloro- 2-((4-fluoro-2- methylphenyl) amino)benzoate | 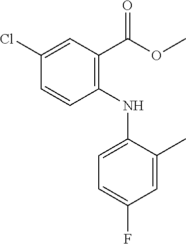 [SMILES: COC(=O)c1cc(Cl)ccc1Nc1ccc(F)cc1C] | MS (m/z) 294.2 (M + H⁺) | methyl 2- bromo-5- chloro- benzoate | -fluoro-2- methyl- aniline |

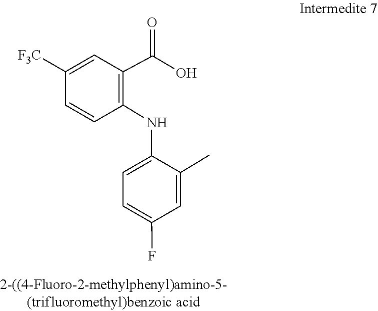 [SMILES: Cc1cc(F)ccc1Nc1ccc(C(F)(F)F)cc1C(=O)O]

To a solution of methyl 2-((4-fluoro-2-methylphenyl)amino)-5-(trifluoromethyl)benzoate (2.3 g, 7.03 mmol in THF (20 mL) under N₂ was added LiOH (1.68 g, 70.3 mmol). The reaction mixture was stirred at 80° C. for 4 h. The reaction mixture was cooled to rt and filtrate was concentrated under vacuum. Crude material was extracted with 100 mL DCM and washed with 50 mL water. Aqueous layer was acidified with 1.5 N HCl 20 mL and extracted with DCM (100 mL) twice. Combined organic layer was dried over sodium sulphate, filtered and concentrated under vacuum to afford the title compound as a yellow solid (2.1 g, 6.7 mmol, 95% yield). MS (m/z) 314.0 (M+H⁺).

Intermediates 8-9 were prepared from the indicated ester by methods analogous to those described for Intermediate 7.

| Int. | Name | Structure | Characterization | Ester |
|---|---|---|---|---|
| 8 | 2-((4-fluoro-2- methylphenyl)amino)- 4- (trifluoromethyl) benzoic acid | 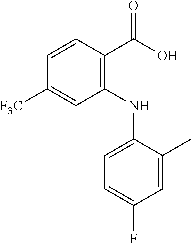 [SMILES: Cc1cc(F)ccc1Nc1cc(C(F)(F)F)ccc1C(=O)O] | MS (m/z) 311.9 (M − H)⁻ | ethyl 2-((4-fluoro-2- methylphenyl)amino)- 4- (trifluoromethyl) benzoate |
| 9 | 5-chloro-2-((4-fluoro-2- methylphenyl)amino) benzoic acid | 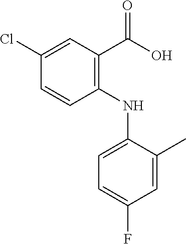 [SMILES: Cc1cc(F)ccc1Nc1ccc(Cl)cc1C(=O)O] | MS (m/z) 280.0 (M + H)⁺ | methyl 5-chloro-2-((4- fluoro-2- methylphenyl)amino) benzoate |

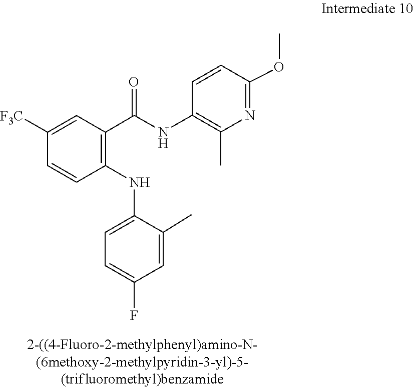 [SMILES: COc1ccc(NC(=O)c2cc(C(F)(F)F)ccc2Nc2ccc(F)cc2C)c(C)n1]

To a solution of 2-((4-fluoro-2-methylphenyl)amino)-5-(trifluoromethyl)benzoic acid (2.1 g, 6.7 mmol), DIPEA (2.34 mL, 13.4 mmol) and HATU (3.82 g, 10.1 mmol) in DMF (20 mL) under nitrogen at RT was added 6-methoxy-2-methylpyridin-3-amine (1.02 g, 7.4 mmol) dropwise over 1 min. The reaction mixture was stirred at RT for 12 h. The reaction mixture was quenched with ice cold water (100 mL) and resulting solid was filtered and dried under vacuum to afford the title compound as a brown solid (2.4 g, 5.5 mmol, 82% yield). MS (m/z) 434.0 (M+H⁺).

Intermediates 11-12 were prepared from the indicated amine and carboxylic acid by methods analogous to those described for Intermediate 10.

| Int. | Name | Structure | Characterization | Amine | acid |
|---|---|---|---|---|---|
| 11 | 2-((4-fluoro-2- methylphenyl)amino)- N-(6-methoxy-2- methylpyridin-3-yl)- 4- (trifluoromethyl) benzamide | 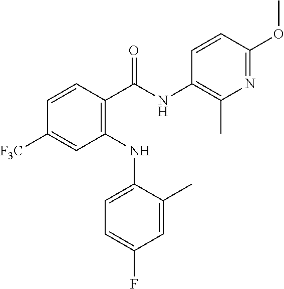 [SMILES: COc1ccc(NC(=O)c2ccc(C(F)(F)F)cc2Nc2ccc(F)cc2C)c(C)n1] | MS (m/z) 433.9 (M + H⁺) | 6-methoxy- 2-methyl- pyridin- 3-amine | 2-((4-fluoro-2- methylphenyl) amino)-4- (trifluoromethyl) benzoic acid |
| 12 | 5-chloro-2-((4-fluoro- 2- methylphenyl)amino)- N-(6-methoxy-2- methylpyridin-3- yl)benzamide | 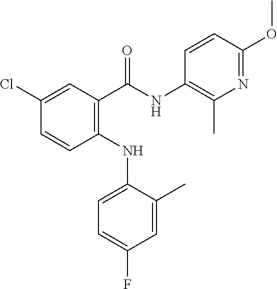 [SMILES: COc1ccc(NC(=O)c2cc(Cl)ccc2Nc2ccc(F)cc2C)c(C)n1] | MS (m/z) 400.0 (M + H⁺) | 6-methoxy- 2-methyl pyridin- 3-amine | 5-chloro-2-((4- fluoro-2- methylphenyl) amino)benzoic acid |

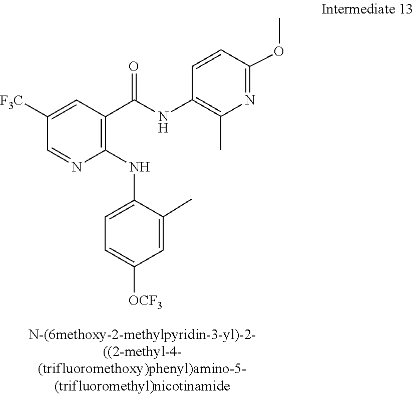 [SMILES: COc1ccc(NC(=O)c2cc(C(F)(F)F)cnc2Nc2ccc(OC(F)(F)F)cc2C)c(C)n1]

A 1 L round bottom flask, fitted with a magnetic stir bar, was charged with 2-chloro-N-(6-methoxy-2-methylpyridin-3-yl)-5-(trifluoromethyl)nicotinamide (37 g, 107 mmol) and 2-methyl-4-(trifluoromethoxy)aniline (30.7 g, 161 mmol). Toluene (500 mL) was added followed by cesium carbonate (69.7 g, 214 mmol). The resulting reaction mixture was purged with nitrogen for 15 minutes before Xantphos (6.19 g, 10.70 mmol) and Pd₂(dba)₃ (4.90 g, 5.35 mmol) were added. The resulting dark brown reaction mixture was stirred at 100° C. for 16 hours. The reaction mixture was allowed to cool to room temperature and filtered through a Celite @bed washing with EtOAc (1 L). The filtrate was concentrated under vacuum and the crude product was purified by flash column chromatography (Biotage, 330 g SiO₂ column, 0-30% EtOAc/petroleum ether over 90 minutes) to give the title compound as a brown solid (35 g, 36.5 mmol, 34.1% yield). MS (m/z) 500.8 (M+H⁺).

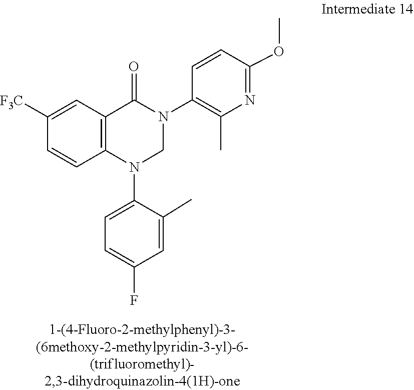 [SMILES: COc1ccc(N2CN(c3ccc(F)cc3C)c3ccc(C(F)(F)F)cc3C2=O)c(C)n1]

To a solution of 2-((4-fluoro-2-methylphenyl)amino)-N-(6-methoxy-2-methylpyridin-3-yl)-5-(trifluoromethyl)benzamide (2.4 g, 5.5 mmol) and Cs₂CO₃ (7.22 g, 22.2 mmol) in acetonitrile (25 mL) under nitrogen at room temperature was added diiodomethane (1.340 mL, 16.61 mmol) dropwise over 5 min. The reaction mixture was stirred at 80° C. for 16 h. The reaction mixture was cooled to rt and filtered through Celite @pad. The filtrate was concentrated onto SiO₂. Purification by flash chromatography on SiO₂ (50 g) with 0-100% EtOAc/petroleum ether as eluant afforded the title compound as a colorless solid (2.0 g, 4.1 mmol, 74% yield). MS (m/z) 446.0 (M+H⁺).

Intermediates 15-17 were prepared from the indicated amide by methods analogous to those described for Intermediate 14.

| Int. | Name | Structure | Characterization | Amide |
|---|---|---|---|---|
| 15 | 1-(4-fluoro-2- methylphenyl)-3-(6- methoxy-2- methylpyridin-3-yl)-7- (trifluoromethyl)-2,3- dihydroquinazolin- 4(1H)-one | 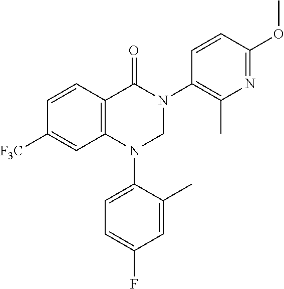 [SMILES: COc1ccc(N2CN(c3ccc(F)cc3C)c3cc(C(F)(F)F)ccc3C2=O)c(C)n1] | MS (m/z) 446.0 (M + H)⁺ | 2-((4-fluoro-2- methylphenyl)amino)- N-(6-methoxy-2- methylpyridin-3-yl)-4- (trifluoromethyl) benzamide |
| 16 | 6-chloro-1-(4-fluoro-2- methylphenyl)-3-(6- methoxy-2- methylpyridin-3-yl)- 2,3-dihydroquinazolin- 4(1H)-one | 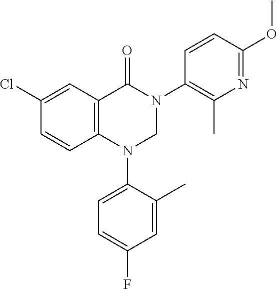 [SMILES: COc1ccc(N2CN(c3ccc(F)cc3C)c3ccc(Cl)cc3C2=O)c(C)n1] | MS (m/z) 412.0 (M + H)⁺ | 5-chloro-2-((4-fluoro- 2- methylphenyl)amino)- N-(6-methoxy-2- methylpyridin-3- yl)benzamide |
| 17 | 3-(6-methoxy-2- methylpyridin-3-yl)-1- (2-methyl-4- (trifluoromethoxy) phenyl)-6- (trifluoromethyl)- 2,3-dihydropyrido[2,3- d]pyrimidin-4(1H)-one | 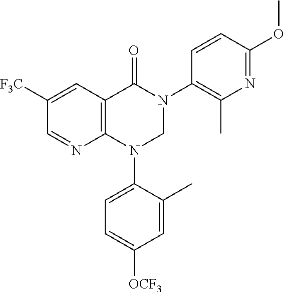 [SMILES: COc1ccc(N2CN(c3ccc(OC(F)(F)F)cc3C)c3ncc(C(F)(F)F)cc3C2=O)c(C)n1] | MS (m/z) 512.8 (M + H)⁺ | N-(6-methoxy-2- methylpyridin-3-yl)-2- ((2-methyl-4- (trifluoromethoxy) phenyl)amino)-5- (trifluoromethyl) nicotinamide |

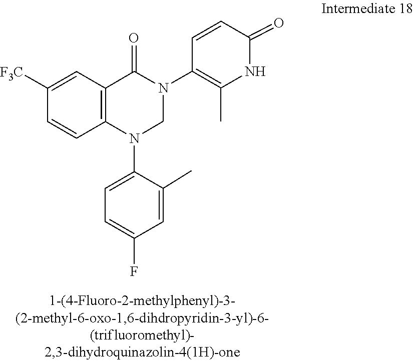 [SMILES: Cc1cc(F)ccc1N1CN(c2ccc(=O)[nH]c2C)C(=O)c2cc(C(F)(F)F)ccc21]

To a solution of 1-(4-fluoro-2-methylphenyl)-3-(6-methoxy-2-methylpyridin-3-yl)-6-(trifluoromethyl)-2,3-dihydroquinazolin-4(1H)-one (0.6 g, 1.4 mmol) in acetonitrile (10 ml-) under nitrogen at room temperature was added iodotrimethylsilane (0.54 g, 2.7 mmol) dropwise over 5 min. The reaction mixture was stirred at 80° C. for 12 h. The reaction mixture was cooled to rt and concentrated under vacuum. The crude residue was dissolved in DCM (100 ml-) and washed with sat. sodium thiosulphate (20 mL). Organic layer was dried over sodium sulphate and concentrated onto Celite S. Purification by reverse phase chromatography on 018 (40 g) with 0-100% gradient with 0.1% formic acid in acetonitrile in 0.1% formic acid in water as eluant afforded clean fractions which were concentrated and the resulting precipitate with filtered, washed with water and dried to afford the title compound as a colorless solid (0.21 g, 0.5 mmol, 36% yield). MS (m/z) 432.1 (M+H⁺).

Intermediates 19-21 were prepared from the indicated intermediate by methods analogous to those described for Intermediate 18.

| Int. | Name | Structure | Characterization | Intermediate |
|---|---|---|---|---|
| 19 | 1-(4-fluoro-2- methylphenyl)-3-(2- methyl-6-oxo-1,6- dihydropyridin-3-yl)-7- (trifluoromethyl)-2,3- dihydroquinazolin- 4(1H)-one | 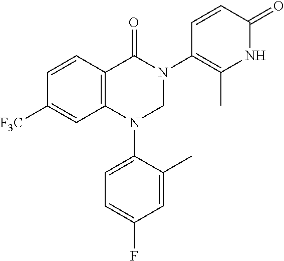 [SMILES: Cc1cc(F)ccc1N1CN(c2ccc(=O)[nH]c2C)C(=O)c2ccc(C(F)(F)F)cc21] | MS (m/z) 432.0 (M + H⁺) | 1-(4-fluoro-2- methylphenyl)-3-(6- methoxy-2- methylpyridin-3-yl)-7- (trifluoromethyl)-2,3- dihydroquinazolin- 4(1H)-one |
| 20 | 6-chloro-1-(4-fluoro-2- methylphenyl)-3-(2- methyl-6-oxo-1,6- dihydropyridin-3-yl)- 2,3-dihydroquinazolin- 4(1H)-one | 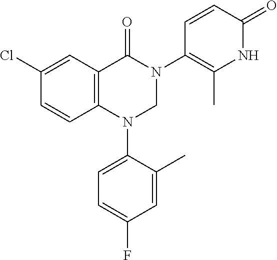 [SMILES: Cc1cc(F)ccc1N1CN(c2ccc(=O)[nH]c2C)C(=O)c2cc(Cl)ccc21] | MS (m/z) 398.0 (M + H⁺) | 6-chloro-1-(4-fluoro- 2-methylphenyl)-3-(6- methoxy-2- methylpyridin-3-yl)- 2,3- dihydroquinazolin- 4(1H)-one |
| 21 | 1-(2-methyl-4- (trifluoromethoxy) phenyl)- 3-(2-methyl-6-oxo- 1,6-dihydropyridin-3- yl)-6-(trifluoromethyl)- 2,3-dihydropyrido[2,3- d]pyrimidin-4(1H)-one | 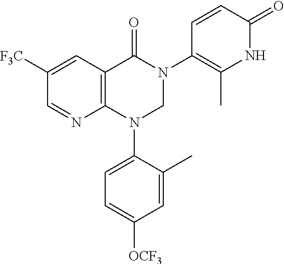 [SMILES: Cc1cc(OC(F)(F)F)ccc1N1CN(c2ccc(=O)[nH]c2C)C(=O)c2cc(C(F)(F)F)cnc21] | MS (m/z) 499.2 (M + H⁺) | 3-(6-methoxy-2- methylpyridin-3-yl)-1- (2-methyl-4- (trifluoromethoxy) phenyl)-6- (trifluoromethyl)-2,3- dihydropyrido[2,3- d]pyrimidin-4(1H)-one |

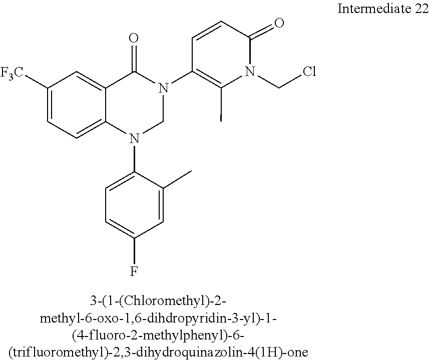 [SMILES: Cc1cc(F)ccc1N1CN(c2ccc(=O)n(CCl)c2C)C(=O)c2cc(C(F)(F)F)ccc21]

Chloromethyl carbonochloridate (0.452 ml, 5.09 mmol) was added dropwise to a suspension of 1-(4-fluoro-2-methylphenyl)-3-(2-methyl-6-oxo-1,6-dihydropyridin-3-yl)-6-(trifluoromethyl)-2,3-dihydroquinazolin-4(1H)-one (0.878 g, 2.035 mmol) and DABCO (0.183 g, 1.628 mmol) in Ethyl acetate (16.23 ml) and DMF (1.623 ml) under N₂. The reaction was stirred at 60° C. for ˜6 hr and then at RT for 2.5 days. The reaction was quenched slowly with sat. NaHCO₃ (20 mL), extracted with EtOAc and DCM (2X). The combined organic extracts were dried over Na₂SO₄ and concentrated. The residue was triturated with a solution of 1:2/EtOAc: heptane to give the title compound as an off-white solid (0.749 g, 1.561 mmol, 77% yield). MS (m/z) 480.3 (M+H⁺).

Intermediates 23-25 were prepared from the indicated amide by methods analogous to those described for Intermediate 22.

| Int. | Name | Structure | Characterization | Amide |
|---|---|---|---|---|
| 23 | 3-(1-(chloromethyl)- 2-methyl-6-oxo-1,6- dihydropyridin-3-yl)- 1-(4-fluoro-2- methylphenyl)-7- (trifluoromethyl)-2,3- dihydroquinazolin- 4(1H)-one | 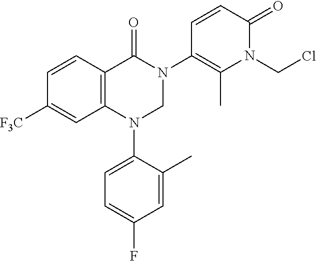 [SMILES: Cc1cc(F)ccc1N1CN(c2ccc(=O)n(CCl)c2C)C(=O)c2ccc(C(F)(F)F)cc21] | MS (m/z) 480.0 (M + H)⁺ | 1-(4-fluoro-2- methylphenyl)-3-(2- methyl-6-oxo-1,6- dihydropyridin-3- yl)-7- (trifluoromethyl)- 2,3- dihydroquinazolin- 4(1H)-one |
| 24 | 6-chloro-3-(1- (chloromethyl)-2- methyl-6-oxo-1,6- dihydropyridin-3-yl)- 1-(4-fluoro-2- methylphenyl)-2,3- dihydroquinazolin- 4(1H)-one | 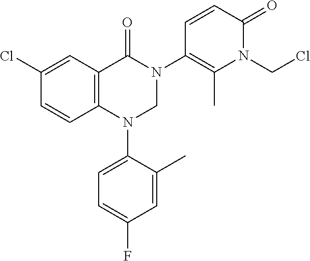 [SMILES: Cc1cc(F)ccc1N1CN(c2ccc(=O)n(CCl)c2C)C(=O)c2cc(Cl)ccc21] | MS (m/z) 446.0 (M + H)⁺ | 6-chloro-1-(4- fluoro-2- methylphenyl)-3-(2- methyl-6-oxo-1,6- dihydropyridin-3- yl)-2,3- dihydroquinazolin- 4(1H)-one |
| 25 | 3-(1-(chloromethyl)- 2-methyl-6-oxo-1,6- dihydropyridin-3-yl)- 1-(2-methyl-4- (trifluoromethoxy) phenyl)-6- (trifluoromethyl)-2,3- dihydropyrido[2,3- d]pyrimidin-4(1H)- one | 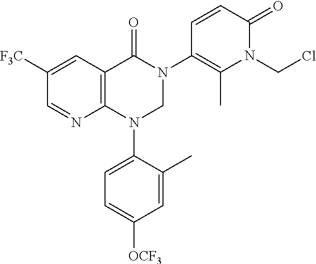 [SMILES: Cc1cc(OC(F)(F)F)ccc1N1CN(c2ccc(=O)n(CCl)c2C)C(=O)c2cc(C(F)(F)F)cnc21] | MS (m/z) 546.8 (M + H)⁺ | 1-(2-methyl-4- (trifluoromethoxy) phenyl)-3-(2-methyl- 6-oxo-1,6- dihydropyridin-3- yl)-6- (trifluoromethyl)- 2,3- dihydropyrido[2,3- d]pyrimidin-4(1H)- one |

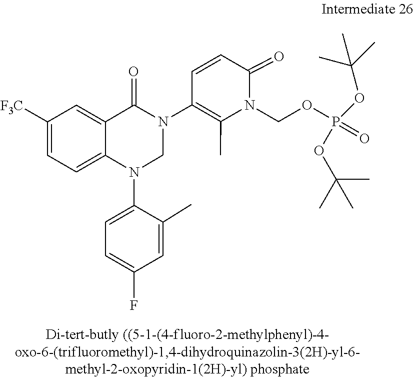 [SMILES: Cc1cc(F)ccc1N1CN(c2ccc(=O)n(COP(=O)(OC(C)(C)C)OC(C)(C)C)c2C)C(=O)c2cc(C(F)(F)F)ccc21]

DMF (7.29 ml) was added to a mixture of 3-(1-(chloromethyl)-2-methyl-6-oxo-1,6-dihydropyridin-3-yl)-1-(4-fluoro-2-methylphenyl)-6-(trifluoromethyl)-2,3-dihydroquinazolin-4(1H)-one (0.749 g, 1.561 mmol), potassium di-tert-butyl phosphate (0.581 g, 2.341 mmol) and tetrabutylammonium iodide (0.029 g, 0.078 mmol) under N₂ and the reaction was stirred at 70° C. for 3 hr. The reaction was cooled to RT and quenched slowly with ice water (40 mL). The reaction was extracted with EtOAc, washed with water (2X), dried over Na₂SO₄ and concentrated to give the title compound as a foam (0.846 g, 1.294 mmol, 83% yield. MS (m/z) 542.3 (M-112)⁺.

Intermediates 27-29 were prepared from the indicated intermediate by methods analogous to those described for Intermediate 26.

| Int. | Name | Structure | Characterization | Intermediate |
|---|---|---|---|---|
| 27 | di-tert-butyl ((5-(1- (4-fluoro-2- methylphenyl)-4- oxo-7- (trifluoromethyl)-1,4- dihydroquinazolin- 3(2H)-yl)-6-methyl- 2-oxopyridin-1(2H)- yl)methyl) phosphate | 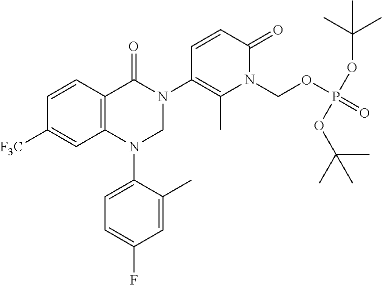 | MS (m/z) 542.0 (M − 112)⁺ | 3-(1- (chloromethyl)-2- methyl-6-oxo-1,6- dihydropyridin-3- yl)-1-(4-fluoro-2- methylphenyl)-7- (trifluoromethyl)- 2,3- dihydroquinazolin- 4(1H)-one |
| 28 | di-tert-butyl ((5-(6- chloro-1-(4-fluoro-2- methylphenyl)-4- oxo-1,4- dihydroquinazolin- 3(2H)-yl)-6-methyl- 2-oxopyridin-1(2H)- yl)methyl) phosphate | 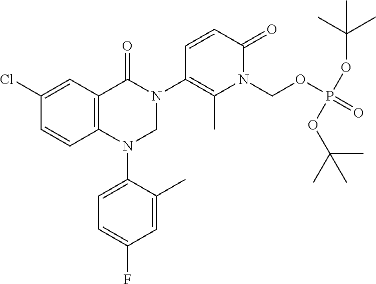 | MS (m/z) 508.0 (M − 112)⁺ | 6-chloro-3-(1- (chloromethyl)-2- methyl-6-oxo-1,6- dihydropyridin-3- yl)-1-(4-fluoro-2- methylphenyl)-2,3- dihydroquinazolin- 4(1H)-one |
| 29 | di-tert-butyl ((6- methyl-5-(1-(2- methyl-4- (trifluoromethoxy) phenyl)-4-oxo-6- (trifluoromethyl)-1,4- dihydropyrido[2,3- d]pyrimidin-3(2H)- yl)-2-oxopyridin- 1(2H)-yl)methyl) phosphate | 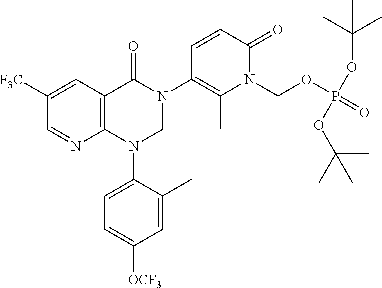 | MS (m/z) 609.0 (M − 112)⁺ | 3-(1- (chloromethyl)-2- methyl-6-oxo-1,6- dihydropyridin-3- yl)-1-(2-methyl-4- (trifluoromethoxy) phenyl)-6- (trifluoromethyl)- 2,3- dihydropyrido[2,3- d]pyrimidin-4(1H)- one |

### Example 1

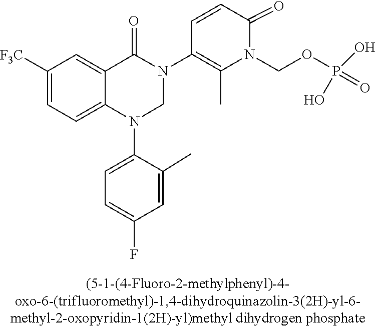 [SMILES: Cc1cc(F)ccc1N1CN(c2ccc(=O)n(COP(=O)(O)O)c2C)C(=O)c2cc(C(F)(F)F)ccc21]

Acetic acid (0.5 ml, 8.73 mmol) was added dropwise to a suspension of di-tert-butyl ((5-(1-(4-fluoro-2-methylphenyl)-4-oxo-6-(trifluoromethyl)-1,4-dihydroquinazolin-3(2H)-yl)-6-methyl-2-oxopyridin-1(2H)-yl)methyl) phosphate (0.3 g, 0.459 mmol) in acetonitrile (1.5 ml) and water (1.5 ml) at RT under N₂ and the reaction was stirred at 70° C. for 3 hr. The reaction was cooled and concentrated. The foam residue was rinsed with water. The residue was purified by reverse phase (EZ Prep Isco, C18 Aq 15.5 g Gold column, 30-80% gradient, acetonitrile with 0.1% formic acid/water with 0.1% formic acid, 30 mL/min flow rate, 12.3 min overall run time) to give the title compound as a white solid (64.7 mg, 0.120 mmol, 26.0% yield). ¹H NMR (400 MHz, DMSO-d₆) δ: 11.60 (br s, 2H), 8.09 (d, J=2.4 Hz, 1H), 7.66 (br d, J=8.3 Hz, 1H), 7.52-7.38 (m, 2H), 7.37-7.28 (m, 1H), 7.26-7.15 (m, 1H), 6.43-6.30 (m, 2H), 5.80-5.60 (m, 2H), 5.55 (d, J=9.3 Hz, 0.6H), 5.1-5.2 (m, 0.8H), 4.75 (br d, J=9.3 Hz, 0.6H), 2.3-2.4 (m, 3H), 2.25 (br s, 3H). MS (m/z) 542.0 (M+H⁺).

Examples 2-4 were prepared from the indicated Intermediate by methods analogous to those described for Example 1.

| Ex. | Name | Structure | Characterization | Intermediate |
|---|---|---|---|---|
| 2 | (5-(1-(4-fluoro-2- methylphenyl)-4- oxo-7- (trifluoromethyl)- 1,4- dihydroquinazolin- 3(2H)-yl)-6- methyl-2- oxopyridin-1(2H)- yl)methyl dihydrogen phosphate | 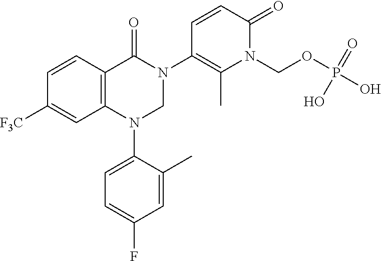 [SMILES: Cc1cc(F)ccc1N1CN(c2ccc(=O)n(COP(=O)(O)O)c2C)C(=O)c2ccc(C(F)(F)F)cc21] | ¹H NMR (400 MHz, DMSO-d₆) δ: 8.07 (d, J = 8.0 Hz, 1H), 7.46-7.37 (m, 2H), 7.35-7.13 (m, 5H), 6.47-6.25 (m, 2H), 5.57 (br s, 2H), 5.51 (br s, 0.5H), 5.13 (br s, 1H), 4.74 (br s, 0.5H), 2.44-2.31 (m, 3H), 2.24 (s, 3H) MS (m/z) 542.0 (M + H)⁺ | di-tert-butyl ((5-(1- (4-fluoro-2- methylphenyl)-4- oxo-7- (trifluoromethyl)- 1,4- dihydroquinazolin- 3(2H)-yl)-6-methyl- 2-oxopyridin-1(2H)- yl)methyl) phosphate |
| 3 | (5-(6-chloro-1-(4- fluoro-2- methylphenyl)-4- oxo-1,4- dihydroquinazolin- 3(2H)-yl)-6- methyl-2- oxopyridin-1(2H)- yl)methyl dihydrogen phosphate | 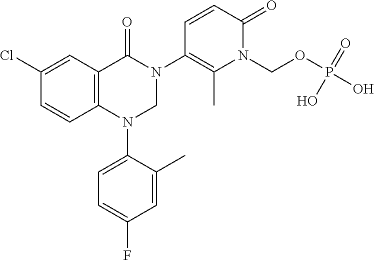 [SMILES: Cc1cc(F)ccc1N1CN(c2ccc(=O)n(COP(=O)(O)O)c2C)C(=O)c2cc(Cl)ccc21] | ¹H NMR (400 MHz, DMSO-d₆) δ: 7.80 (d, J = 2.6 Hz, 1H), 7.46 (d, J = 9.8 Hz, 1H), 7.40 (dd, J = 8.8, 2.6 Hz, 1H), 7.36- 7.27 (m, 2H), 7.22-7.12 (m, 1H), 6.39 (d, J = 9.6 Hz, 1H), 6.35-6.17 (m, 1H), 5.83-5.69 (m, 2H), 5.46 (brs, 0.6H), 5.21- 4.97 (m, 0.8H), 4.69 (br s, 0.6H), 2.43-2.28 (m, 3H), 2.23 (s, 3H) MS (m/z) 508.0 (M + H)⁺ | di-tert-butyl ((5-(6- chloro-1-(4-fluoro- 2-methylphenyl)-4- oxo-1,4- dihydroquinazolin- 3(2H)-yl)-6-methyl- 2-oxopyridin-1(2H)- yl)methyl) phosphate |
| 4 | (6-methyl-5-(1-(2- methyl-4- (trifluoromethoxy) phenyl)-4-oxo-6- (trifluoromethyl)- 1,4- dihydropyrido[2,3- d]pyrimidin-3(2H)- yl)-2-oxopyridin- 1(2H)-yl)methyl dihydrogen phosphate | 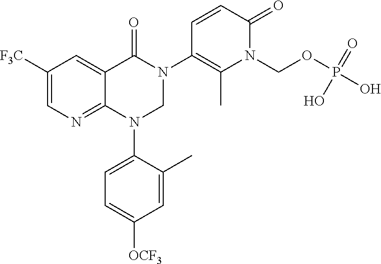 [SMILES: Cc1cc(OC(F)(F)F)ccc1N1CN(c2ccc(=O)n(COP(=O)(O)O)c2C)C(=O)c2cc(C(F)(F)F)cnc21] | ¹H NMR (400 MHz, DMSO-d₆) δ: 8.60 (s, 1H), 8.32 (d, J = 2.40 Hz, 1H), 7.55-7.15 (m, 6H), 6.30 (t, J = 10.00 Hz, 1H), 5.70- 5.50 (m, 2.5H), 5.35 (d, J = 10 Hz, 0.5H), 5.22 (d, J = 10 Hz, 0.5H) 4.92 (d, J = 9.6 Hz, 0.5H), 2.42 (s, 3H), 2.24 (d, J = 6.8 Hz, 3H) MS (m/z) 609.0 (M + H)⁺ | di-tert-butyl ((6- methyl-5-(1-(2- methyl-4- (trifluoromethoxy) phenyl)-4-oxo-6- (trifluoromethyl)- 1,4- dihydropyrido[2,3- d]pyrimidin-3(2H)- yl)-2-oxopyridin- 1(2H)-yl)methyl) phosphate |

### Biological Assays

The Naj1.8 Inhibitor compounds or pharmaceutically acceptable salts thereof of the invention are useful for treatment of pain, pain disorders or conditions, pain-related disorders or conditions or pain caused by diseases, respectively, such as those defined throughout the instant application.

The biological activity of the compounds of the invention can be determined using suitable assays, such as those measuring such inhibition and those evaluating the ability of the compounds to inhibit voltage gated sodium channel Naᵥ 1.8 in vitro or in animal models of infection.

### Biological Assay Example 1

Human embryonic kidney 293 cells (HEK293) expressing human Naᵥ1.8, human Naᵥβ1 and human TREK1 (HEK293-Naᵥ1.8) were grown at 37° C., 5% CO₂ in 150 cm² flasks. HEK293-Naᵥ1.8 were passaged every 2-3 days into T175 cell culture flasks when confluency reached 80-90%.

Pharmacological assessment of the compounds of the invention was performed using HEK293-Nav1.8 in combination with an assay developed on the QPatch 48 HTX electrophysiological system. HEK293-Naᵥ1.8 were prepared on the day of use by removing culture media, washing in DPBS, adding Accutase (2 ml to cover the surface, aspirate 1 ml then 1.5 min at 37° C.) followed by addition of CHO-SFM II to stop the enzyme digestion and in order to obtain a suspension of 3×10⁶ cell/mL.

Compound was prepared in an extracellular solution of the following composition: NaCl (145 mM), KCl (4 mM), CaCl₂ (2 mM), MgCl₂ (2 mM), HEPES (1 mM), Glucose (10 mM), pH 7.4 with NaOH Osmolality 300 mOsM/L. The intracellular solution was used of the following composition: CsF (115 mM), CsCl (20 mM), NaCl (5 mM), EGTA (10 mM), HEPES (10 mM), Sucrose (20 mM), pH 7.2 with CsOH Osmolality 310 mOsm/L.

Utilizing the voltage-clamp mode in the QPatch 48 HTX system a half inactivation state voltage protocol (V₁/2) was used to determine pharmacological activity of compounds of the invention at Naᵥ1.8 ion channels. A V_{1/2} protocol was utilized with the following voltage steps: a holding voltage of −100 mV was established followed by a 20 ms voltage step to 0 mV (P1), followed by an inactivating voltage step at −46 mV for 8 seconds, followed by a step to −100 mV for 20 ms, before a 20 ms step to 0 mV (P2) before returning to the holding voltage of −100 mV. This voltage protocol was repeated at a frequency of 0.07 Hz., current magnitude was quantified at the P2 step throughout the recording. Inhibition of the measured current amplitude with the compounds of the invention was analyzed by fitting a 6-8 point dose-response curve allowing determination of the fifty percent inhibition concentration (IC₅₀). Within the QPatch HTX software, P2 current was normalized according to measurements made at baseline after compound and after positive reference compound and fit to the following equation:

$$n . {I}_{CPD} = Normalized Current = \frac{( Input - Baseline )}{( FullResponse - Baseline )}$$

To assess current run-down over the course of the experiment vehicle-only wells were utilized and the normalized current with vehicle-only (n.I_{VEH}) was determined. To correct the compound response for run-down, the currents were corrected according the following formula:

$$n . {I}_{RD \_ Correct} = \frac{( n . {I}_{CPD} - n . {I}_{VEH} )}{( 1 - n . {I}_{VEH} )}$$

Compounds of the invention and the corresponding parent compounds (see Table 1A for structures) were tested for activity against Nav1.8 sodium channels in the above assay in one or more experimental runs and the results are shown in Table 1 below. Potency of the compounds of the invention is reported as a pIC50 value. The pIC50 value is the negative log of the IC50 value, wherein the IC50 value is the half maximal inhibitory concentration measured in molar (M). Potency of the compounds of the invention is compared to the potency of the parent compound. For compounds tested in more than one experimental run, the pIC50 value is reported as an average.

TABLE 1

| | |
|---|---|
| Compounds of the Invention | Parent Compounds |

| Compound | [Nav1.8] | Parent | [Nav1.8] |
|---|---|---|---|
| Example No. | pIC50 | Compound No. | pIC50 |
| 1 | 6.8 | 1A | 8 |
| 2 | 6.6 | 2A | 7.7 |
| 3 | 6.9 | 3A | 8 |
| 4 | 6.1 | 4A | 7.5 |

TABLE 1A

| Compound | | Parent | |
|---|---|---|---|
| Example | | Compound | |
| No. | Structure | No. | Structure |
| 1 | 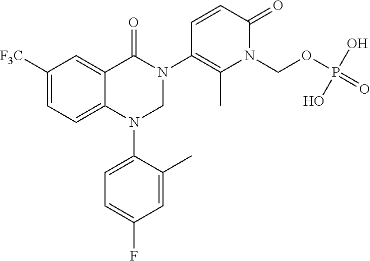 [SMILES: Cc1cc(F)ccc1N1CN(c2ccc(=O)n(COP(=O)(O)O)c2C)C(=O)c2cc(C(F)(F)F)ccc21] | 1A | 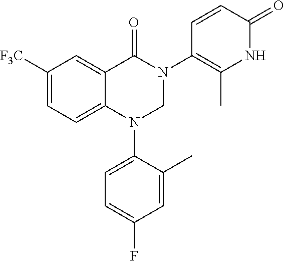 [SMILES: Cc1cc(F)ccc1N1CN(c2ccc(=O)[nH]c2C)C(=O)c2cc(C(F)(F)F)ccc21] |
| 2 | 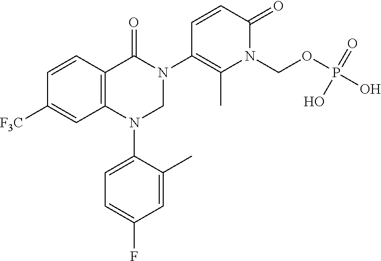 [SMILES: Cc1cc(F)ccc1N1CN(c2ccc(=O)n(COP(=O)(O)O)c2C)C(=O)c2ccc(C(F)(F)F)cc21] | 2A | 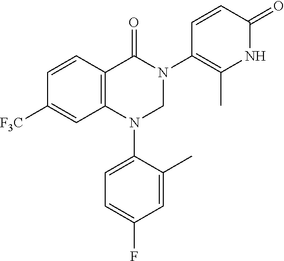 [SMILES: Cc1cc(F)ccc1N1CN(c2ccc(=O)[nH]c2C)C(=O)c2ccc(C(F)(F)F)cc21] |
| 3 | 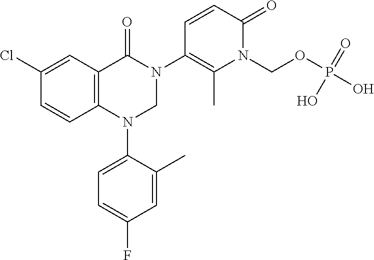 [SMILES: Cc1cc(F)ccc1N1CN(c2ccc(=O)n(COP(=O)(O)O)c2C)C(=O)c2cc(Cl)ccc21] | 3A | 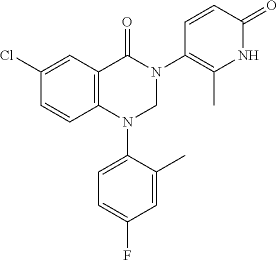 [SMILES: Cc1cc(F)ccc1N1CN(c2ccc(=O)[nH]c2C)C(=O)c2cc(Cl)ccc21] |
| 4 | 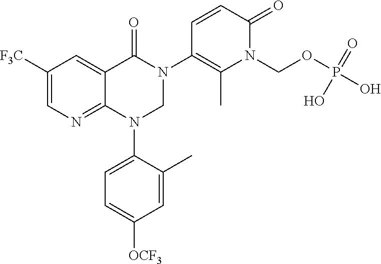 [SMILES: Cc1cc(OC(F)(F)F)ccc1N1CN(c2ccc(=O)n(COP(=O)(O)O)c2C)C(=O)c2cc(C(F)(F)F)cnc21] | 4A | 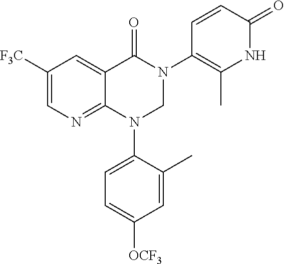 [SMILES: Cc1cc(OC(F)(F)F)ccc1N1CN(c2ccc(=O)[nH]c2C)C(=O)c2cc(C(F)(F)F)cnc21] |

### Biological Assay Example 2

Kinetic solubility measurement using Charged Aerosol Detector (CAD). The aqueous kinetic solubility at pH 7.4 was determined by measuring the concentration of solute in solution after precipitation from DMSO stock solution. The DMSO stock solution was diluted 20-fold with phosphate buffered saline (PBS) pH 7.4 and the solubility of the compound was measured after 1 hour equilibration at room temperature by HPLC-CAD. Calibration standards of Ketoconazole and Primidone were prepared by serial dilutions in DMSO at concentrations ranging from 0.016 to 4.5 mg/ml to produce the calibration curve used to determine the solubility of the compounds as previously described in Max W. Robinson et al, Use of Calculated Physicochemical Properties to Enhance Quantitative Response When Using Charged Aerosol Detection, Anal. Chem., 2017, 89 (3), pp 1772-1777, which is herein incorporated by reference.

CAD solubility of the compounds of the invention and the corresponding parent compounds was measured as described above and the results are shown in Table 2 below.

TABLE 2

| | |
|---|---|
| Prodrug Compounds | |
| of the Invention | Parent Compounds |

| Compound | CAD | Parent | CAD |
|---|---|---|---|
| Example | solubility^{a} | Compound | solubility^{b} |
| No. | (μg/mL) | No. | (μg/mL) |
| 1 | >260 | 1A | 48 |
| 2 | >193 | 2A | 51 |
| 3 | 102 | 3A | 54 |
| 4 | 181 | 4A | 87 |

^{a}CAD solubility was measured in multiple experimental runs and the data reported as an average for Compound Example Nos. 1 and 3. CAD solubility was measured in one experimental run for Compound Example Nos. 2 and 4.
^{b}CAD solubility is reported as the average of multiple experimental runs.

### Biological Assay Example 3: Rat IV/PO Study

An in vivo rat pharmacokinetic study was conducted to determine whether the prodrug compounds of the invention are converted to the respective parent compound upon administration. The rat pharmacokinetic study was conducted with a crossover design on two study days with a one-day recovery period between each study day. Three male, dual catheterized (femoral vein and carotid artery) Han Wistar rats were used for the study. Each rat was also implanted with a gastric catheter for oral dose administration. Rats were dosed at 1 mg/kg by a 60 minute intravenous (IV) infusion (femoral vein cannula), then subsequently oral dosed at 2 mg/kg via the gastric cannula, with 48 hours between dosing sessions. Dose solutions of the compound of Example 3 were prepared in 20% Cavitron/5% DMSO/75% water (IV) and in 6% Cavitron/5% DMSO/89% water (PO) without pH adjustment. The dose solutions were filtered using a 0.22 μ filter. The pH of the final dosing solutions was 6.0.

During the intravenous study leg, blood samples were collected from the carotid artery catheter at target times of 15, 30, 60 (end of infusion), 65, 75, 90, 120, 240, 360, 480, 720, and 1440 minutes following the initiation of the intravenous infusion of the compound of Example 3. During the oral study leg, blood samples were collected prior to dosing and at target times of 15, 30, 60, 90, 120, 180, 240, 360, 480, 720, and 1440 minutes following oral administration. Blood samples, 100 μL, were mixed with 100 μL phosphatase inhibitor, a 50 μL aliquot of the blood and inhibitor mixture was transferred to a non-heparinized tube and stored at approximately −80° C. until analyzed. The concentrations in the filtered dose solutions were confirmed by preparing stepwise dilutions first into 50% aqueous acetonitrile with 0.1% formic acid then into heparinized male Wistar Han blood:inhibitor to achieve determined nominal concentrations. Triplicate 50 μL aliquots were removed and were frozen and stored at approximately 80° C. until analyzed by LC-MS/MS as described below.

LC-MS/MS was used to quantify the compound of Example 3 and the corresponding parent compound of Example 3A in the biological samples generated in the above described in vivo study. Samples were prepared by protein precipitation followed by LC-MS/MS analysis employing positive-mode ionization against a set of calibration standards for the compounds prepared in the same matrix. Pharmacokinetic parameters for the study was derived from the concentration versus time profiles. Key pharmacokinetic parameters such as AUC_{0-∞} (extrapolated area under the blood concentration-time curve), AUC₀₋ₜ (area under the blood concentration-time curve to the last time point with quantifiable drug), Cmax (maximum concentration), Tmax (time Cmax is achieved), CL (systemic blood clearance), Vdss (steady-state volume of distribution), MRT (mean residence time), and t_{1/2}(half-life) were determined for the compound of Example 3. The key pharmacokinetic parameters such as AUC_{0-∞}, AUC₀₋ₜ, Cmax, Tmax, MRT, and t_{1/2} (half-life) were determined for the parent compound Example 3A. Descriptive statistical data of pharmacokinetic parameters were calculated, including the mean and standard deviation (SD) using Microsoft Excel.

The data are shown below in Tables 3A and 3B. Data are reported as mean±SD (N=3).

TABLE 3A

| | | Route | |
|---|---|---|---|
| | Parameter | Intravenous | Oral |
| Compound of | Dose (mg/kg) | 0.87 ± 0.01 | 1.7 ± 0.0 |
| Example 3 | Cmax (ng/mL) | 260 ± 24 | There were no |
| (Prodrug) | Tmax (h)^{b} | 1.0, 0.25, 1.0 | quantifiable |
| | Half-life (h)^{b} | 0.42, 3.8, 4.8 | concentrations |
| | MRT (h)^{b} | 0.14, 0.87, 1.2 | following oral |
| | CL (mL/min/kg) | 58 ± 9 | administration |
| | Vdss (L/kg)^{b} | 0.44, 3.6 3.8 | |
| | AUC₀₋ₜ (μg · h/mL) | 0.24 ± 0.04 | |
| | AUC_{0-∞} (μg · h/mL) | 0.25 ± 0.03 | |
| | Bioavailability (%) | | |

^{b}Values listed individually due to variability

TABLE 3B

| | | Route | |
|---|---|---|---|
| | Parameter | Intravenous | Oral |
| Compound of | Dose (mg/kg) | | |
| Example 3A | Cmax (ng/mL) | 170 ± 26 | 170 ± 41 |
| (Parent Compound) | Tmax (h) | 1.1 ± 0.05 | 1.2 ± 0.3 |
| | Half-life (h) | 3.7 ± 0.4 | 3.4 ± 0.5 |
| | MRT (h) | 5.0 ± 0.3 | 7.0 ± 0.2 |
| | CL (mL/min/kg) | | |
| | Vdss (L/kg) | | |
| | AUC₀₋ₜ (μg · h/mL) | 0.58 ± 0.06 | 1.3 ± 0.1 |
| | AUC_{0-∞} (μg · h/mL) | 0.63 ± 0.06 | 1.4 ± 0.1 |
| | Bioavailability (%) | | |

It is to be understood that the invention is not limited to the embodiments illustrated hereinabove and the right is reserved to the illustrated embodiments and all modifications coming within the scope of the following claims.

1.-41. (canceled)

42. A compound of formula (I): 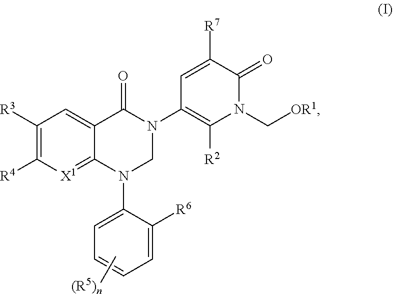
or a pharmaceutically acceptable salt thereof,
wherein:
X¹ is N or CH;
R¹ is —P(O)(OH)₂;
R² is hydrogen, -(C₁₋₆)alkyl, —NR^{a}R^{b}, halo, or -(C₁₋₆)haloalkyl;
each of R³ and R⁴ is independently hydrogen, halo, cyano, —NR^{a}R^{b}, -(C₁₋₆)alkyl, -(C₁₋₆)haloalkyl, —O-(C₁₋₆)alkyl, or —O-(C₁₋₆)haloalkyl;
each R⁶ is independently halo, -(C₁₋₆)alkyl, —O(C₁₋₆)alkyl, or —O(C₁₋₆)haloalkyl;
R⁶ is hydrogen or -(C₁₋₆)alkyl;
R⁷ is hydrogen, -(C₁₋₆)alkyl, halo, or -(C₁₋₆)haloalkyl;
each of R^{a} and R^{b} is independently hydrogen or -(C₁₋₆)alkyl; and
n is 0, 1, or 2.

43. The compound according to claim 42, wherein X¹ is N.

44. The compound according to claim 42, wherein X¹ is CH.

45. The compound according to claim 42, wherein R² is -(C₁₋₆)alkyl.

46. The compound according to claim 42, wherein each of R³ and R⁴ is independently hydrogen, halo, or -(C₁₋₆)haloalkyl.

47. The compound according to claim 46, wherein each of R³ and R⁴ is hydrogen, —Cl, or —CF₃.

48. The compound according to claim 42, wherein one of R³ and R⁴ is hydrogen and the other of R³ and R⁴ is halo or -(C₁₋₆)haloalkyl.

49. The compound according to claim 48, wherein one of R³ and R⁴ is hydrogen and the other of R³ and R⁴ is —Cl or —CF₃.

50. The compound according to claim 42, wherein each R⁵ is independently halo or —O(C₁₋₆)haloalkyl.

51. The compound according to claim 50, wherein each R⁵ is independently —F or —OCF₃.

52. The compound according to claim 42, wherein R⁶ is -(C₁₋₆)alkyl.

53. The compound according to claim 52, wherein R⁶ is —CH₃, —CH₂CH₃, or —CH(CH₃)₂.

54. The compound according to claim 42, wherein:
X¹ is N;
R¹ is —PO(OH)₂;
R² is —CH₃;
one of R³ and R⁴ is hydrogen and the other of R³ and R⁴ is halo or -(C₁₋₆)haloalkyl;
R⁵ is halo or —O(C₁₋₆)haloalkyl;
R⁶ is -(C₁₋₆)alkyl;
R⁷ is hydrogen; and
n is 1.

55. The compound according to claim 42, being a pharmaceutically acceptable salt of the compound of formula (I), wherein:
R¹ is —P(O)(OH)O⁻M⁺, —PO(O⁻)₂·2M⁺, or —PO(O⁻)₂·D²⁺;
each M⁺ is independently a pharmaceutically acceptable monovalent cation; and
D²⁺ is a pharmaceutically acceptable divalent cation.

56. A compound selected from the group consisting of:
(5-(1-(4-Fluoro-2-methylphenyl)-4-oxo-6-(trifluoromethyl)-1,4-dihydroquinazolin-3(2H)-yl)-6-methyl-2-oxopyridin-1(2H)-yl)methyl dihydrogen phosphate;
(5-(1-(4-fluoro-2-methylphenyl)-4-oxo-7-(trifluoromethyl)-1,4-dihydroquinazolin-3(2H)-yl)-6-methyl-2-oxopyridin-1(2H)-yl)methyl dihydrogen phosphate;
(5-(6-chloro-1-(4-fluoro-2-methylphenyl)-4-oxo-1,4-dihydroquinazolin-3(2H)-yl)-6-methyl-2-oxopyridin-1(2H)-yl)methyl dihydrogen phosphate; and
(6-methyl-5-(1-(2-methyl-4-(trifluoromethoxy)phenyl)-4-oxo-6-(trifluoromethyl)-1,4-dihydropyrido[2,3-d]pyrimidin-3(2H)-yl)-2-oxopyridin-1(2H)-yl)methyl dihydrogen phosphate,
or a pharmaceutically acceptable salt thereof.

57. A pharmaceutical composition comprising a compound as defined in claim 42, and a pharmaceutically acceptable excipient.

58. The pharmaceutical composition according to claim 57, formulated for intravenous administration.

59. A method of inhibiting a Naᵥ1.8 voltage-gated sodium channel in a subject in need thereof, the method comprising administering to the subject a compound or pharmaceutically acceptable salt thereof or tautomer thereof according to claim 42 or a pharmaceutically acceptable salt thereof.

60. A method of treatment of pain or a pain-associated disease, disorder, or condition in a subject in need thereof, the method comprising administering to the subject a therapeutically effective amount of a compound according to claim 42 or a pharmaceutically acceptable salt thereof.

61. The method according to claim 60, wherein the pain is acute pain or chronic pain.

62. The method according to claim 60, wherein the pain or pain-associated disease, disorder, or condition is pain caused by trauma; pain caused by iatrogenic medical or dental procedures; or pre-operative or post-operative associated pain.

63. The method according to claim 60, wherein the pain or pain-associated disease, disorder, or condition is neuropathic pain, nociceptive pain, inflammatory pain, musculoskeletal pain, visceral pain, or idiopathic pain.

64. The method according to claim 60, wherein the pain or pain-associated disease, disorder or condition is neuropathic pain or chronic neuropathic pain selected from small fiber neuropathy, small fiber-mediated diabetic neuropathy, idiopathic small fiber neuropathy, painful diabetic neuropathy or polyneuropathy.

65. The method according to claim 60, wherein the pain or pain associated disease, disorder, or condition is inflammatory pain selected from osteoarthritis, chronic osteoarthritis pain, or chronic inflammatory demyelinating polyneuropathy.

66. A method of treatment of atrial fibrillation in a subject in need thereof, the method comprising administering to the subject a therapeutically effective amount of a compound according to claim 42 or a pharmaceutically acceptable salt thereof.

67. The method according to claim 60, wherein the subject is human.

68. The method according to claim 66, wherein the subject is human.
